(19) United States
(12) Patent Application Publication
(10) Pub. No.: US 2003/0113724 A1
(43) Pub. Date: Jun. 19, 2003

(54) Packaged microarray apparatus and a method of bonding a microarray into a package

(76) Inventors: Carol T. Schembri, San Mateo, CA (US); Andre B. Chow, Belmont, CA (US); Laurence R. Shea, San Francisco, CA (US)

Correspondence Address: AGILENT TECHNOLOGIES, INC, Legal Department, DL429, Intellectual Property Administration, P.O. Box 7599, Loveland, CO, 80537-0599, US

(21) Appl. No.: 09/978,822

(22) Filed: Oct. 12, 2001

(51) Int. Cl. C12Q 1/68; C12M 1/34; H01L 21/00

(52) U.S. Cl. 435/6; 435/287.2; 438/1

(57) ABSTRACT

A method of bonding a microarray to a package uses optional surface treatments on the microarray and on the package to enhance the adhesion of the microarray to the package using an adhesive. The adhesive bonds to the microarray and the package with sufficient bond strength and flexibility to withstand the stress caused by different expansion rates during exposure to temperature extremes. A method of attaching the microarray to the package uses a plurality of adhesives to provide the bond strength and flexibility. A packaged microarray apparatus comprises a microarray of biological features, a package, and an adhesive bond between the microarray and the package. The apparatus may have surface treatment and/or the adhesive bond may comprise a plurality of adhesives.

### TECHNICAL FIELD

This invention relates to microarrays. In particular, the invention relates to packaged microarrays and materials and methods for attaching a microarray to a package.

### BACKGROUND ART

Microarrays of nucleic acids (DNA or RNA) or proteins are state-of-the-art biological tools used in the investigation and evaluation of genes for analytical, diagnostic, and therapeutic purposes. Microarrays typically comprise a plurality of the molecular species, synthesized or deposited on a glass support or substrate in an array pattern. The support-bound molecular species are called “probes” and function to bind or hybridize with a sample of material under test, called “target” in hybridization experiments. However, some investigators bind the target sample under test to the microarray substrate and put the molecular probes in solution for hybridization. Either of the “targets” or “probes” may be the one that is to be evaluated by the other (thus, either one could be an unknown mixture of nucleic acids or proteins to be evaluated by binding with the other). All of these iterations are within the scope of this discussion herein. In use, the microarray surface is contacted with one or more targets under conditions that promote specific, high-affinity binding of the target to one or more of the probes. The sample solution typically contains radioactively, chemoluminescently or fluorescently labeled molecules that are detectable, so that the hybridized targets and probes are detected with scanning equipment. Molecular array technology offers the potential of using a multitude (hundreds of thousands) of different molecular species to analyze changing mRNA populations.

There are numerous types of substrates used in hybridization assays. One common type of substrate or support used for microarray assays is a siliceous substrate, such as glass. The surface of the substrate typically is chemically prepared or derivatized to enable or facilitate the attachment of the molecular species to the surface of the array substrate for the manufacture of microarrays. Surface derivatizations can differ for immobilization of prepared biological material, such as cDNA, and in situ synthesis of the biological material on the microarray substrate. Surface treatment or derivatization techniques are well known in the art.

A plurality of microarrays may be formed on a larger array substrate or wafer. In order to make optimal use of the substrate, the substrate is diced into a plurality of individual microarray die. A microarray die may be quite small and difficult to handle for processing. Therefore, the individual microarray die is packaged for further handling and processing. For example, the microarray may be processed by subjecting the microarray to a hybridization assay while retained in the package. Typically, the package or housing is made of a plastic that is injection molded into a suitable package. Examples of packages can be found in U.S. Pat. Nos. 6,140,044 and 5,945,334.

However, the bond between the microarray substrate and the package may not be easily achieved and when achieved, may not reliably withstand thermal stresses during a hybridization assay. Some adhesives do not readily bond to some package materials and/or some substrate materials. Further, the difference in the coefficients of thermal expansion (CTEs) of the substrate and package materials puts stress on the adhesive bond when exposed to temperature extremes. The thermal stress can cause the bond to fail or the cured adhesive to crack.

Thus, it would be advantageous to produce a packaged microarray using materials and techniques that withstand thermal stresses. Such materials and techniques would solve the problem of poor adhesion and/or bond reliability in the packaged microarray art.

### SUMMARY OF THE INVENTION

The present invention is a method of bonding a microarray die to a microarray package such that the bond between the die and the package is stable and reliable under thermal stresses when exposed to temperature extremes. In some embodiments, the method employs surface treatment to the microarray and, if necessary, to the package material to enhance their bondability with adhesives. In other embodiments, the method employs the use of a system or combination of adhesives to accommodate the differences in thermal expansion rates of the substrate and package materials during temperature extremes. Further, the present invention is a packaged microarray apparatus having a stable and reliable adhesive bond between a microarray substrate and a plastic package that can withstand thermal stresses caused by high and low temperature extremes.

In one aspect of the invention, a method of bonding a microarray to a microarray package is provided. The method comprises modifying a bonding surface of the microarray to render the modified surface more wettable to an adhesive. The method further comprises attaching the microarray to the package using the adhesive between the modified bonding surface and an attachment surface of the microarray package. Some package materials, such as polypropylene, may not be readily wetted by some adhesives. Therefore, in one or more embodiments, the method optionally further comprises the step of treating the attachment surface of the microarray package to render the treated surface more wettable by the adhesive. The modified and treated surfaces are more wettable by the adhesive than the wettability of the respective surfaces before the surface treatments.

In another aspect of the present invention, a method of attaching a microarray to a package is provided. The method comprises using a combination of adhesives between a bonding surface of the microarray and an attachment surface of the package. The combination of adhesives provides a more intact bond between the microarray and the package relative to any one of the adhesives from the combination used individually to bond the microarray and the package. The adhesive combination has bond strength and flexibility that are both sufficient enough to keep the bond intact or fluid tight with physical stresses caused by different thermal expansion rates of the microarray and the package.

In still another aspect of the invention, a packaged microarray apparatus is provided. The apparatus comprises a microarray having biological features on an array surface of a microarray substrate and having a bonding surface that is modified to render the surface more wettable by an adhesive. The apparatus further comprises a package having an attachment surface, and an adhesive bond between the microarray and the package at the modified surface and the attachment surface. In one or more embodiments, the attachment surface of the package is optionally surface treated also to render the attachment surface more wettable by the adhesive. The modified and treated surfaces are more wettable by adhesives than the wettability of the respective surfaces before the surface treatments.

In yet another aspect of the invention, a packaged microarray apparatus is provided. The apparatus comprises a microarray having biological features on an array surface of a microarray substrate and having a bonding surface, a package having an attachment surface, and an adhesive bond between the microarray and the package. The adhesive bond comprises a combination of adhesives that provides a more intact bond between the microarray and the package relative to any one of the adhesives from the combination used individually to bond the microarray and the package. The adhesive combination has bond strength and flexibility that are both sufficient enough to keep the bond intact with physical stresses caused by different thermal expansion rates of the microarray and the package.

The adhesive system or combination comprises a cured first adhesive layer on either or both of the microarray bonding surface and the package attachment surface. The adhesive system further comprises a cured second adhesive along a periphery of an interface between the microarray and the package. The second adhesive is allowed to wick into the interface before being cured. Depending on the embodiment, the second adhesive wicks between the first cured adhesive layer on the microarray bonding surface and the package attachment surface, the first cured adhesive layer on the package attachment surface and the microarray bonding surface, and the first cured adhesive layers on each of the microarray bonding surface and the package attachment surface. The adhesive system may further comprise a cured third adhesive layer on the other of the surfaces that does not have the cured first adhesive layer. The cured first and third adhesive layers provide the bond strength to their respective bonding surfaces. The cured second adhesive provides the bond strength to the other cured adhesive layers, the microarray bonding surface, and the package attachment surface. One of the adhesives of the combination is more flexible than the others. Preferably, the cured second adhesive is more flexible than the first and third adhesives.

In one or more embodiments of this packaged microarray apparatus, either or both of the microarray bonding surface and the package attachment surface is surface treated or modified to render the surface more wettable by the adhesives of the combination. Each of the modified surface and the treated surface is rendered more wettable by the adhesives than the wettability of the respective surface before the surface treatments.

Advantageously, the present invention provides a strong and reliable adhesive bond between a microarray and a package that withstands thermal stresses to which the adhesive bond is exposed, especially the high and low temperature extremes of a hybridization assay. In some embodiments, the adhesive bond advantageously provides a fluid tight seal between the microarray and the package that remains intact even after the thermal stress. Certain embodiments of the present invention have other advantages in addition to and in lieu of the advantages described hereinabove. These and other features and advantages of the invention are detailed below with reference to the following drawings.

### BRIEF DESCRIPTION OF THE DRAWINGS

The various features and advantages of the present invention may be more readily understood with reference to the following detailed description taken in conjunction with the accompanying drawings, where like reference numerals designate like structural elements, and in which:

FIG. 1A illustrates a perspective view of a example of a microarray die having a plurality of biological features bound thereto that is taken from an array substrate or wafer of FIG. 1C.

FIG. 1B illustrates a perspective view of another example of a microarray die having a plurality of sets of biological features bound thereto that are fabricated individually or may be taken from the array substrate of FIG. 1C.

FIG. 1C illustrates a perspective view of an array substrate or wafer having a larger plurality of sets of biological features arranged thereon according to the invention and includes an exploded view of a set of biological features.

FIG. 2A illustrates a perspective view of a preferred embodiment of a microarray package to house the microarray example of FIG. 1A.

FIG. 2B illustrates a perspective view of another embodiment of a microarray package to house the microarray example of FIG. 1A.

FIG. 2C illustrates a perspective view of still another embodiment of a microarray package to house the microarray example of FIG. 1A.

FIG. 3A illustrates a flow chart of a method of bonding a microarray in a package according to the present invention.

FIG. 3B illustrates a flow chart of the attachment of the microarray to the package according to the present invention.

FIG. 3C is a cross sectional view of a packaged microarray apparatus according to the present invention using the preferred package type of FIG. 2A.

FIG. 3D is a cross sectional view of a packaged microarray apparatus according to the present invention using the package type of FIG. 2B.

FIG. 4A illustrates a flow chart of a preferred embodiment of a method of attaching a populated microarray to a package.

FIG. 4B illustrates a cross sectional view through a packaged microarray apparatus of the present invention that was assembled using the preferred package type of FIG. 2A and comprises a preferred embodiment of an adhesive bond.

FIG. 4C illustrates a magnified cross sectional view of an adhesive bond and seal at a microarray/package interface of a packaged microarray apparatus of the present invention using either package type of FIGS. 2A or 2C.

FIG. 4D illustrates a cross sectional view through a packaged microarray apparatus of the present invention that was assembled using the package type of FIG. 2B and comprises an alternative embodiment of the adhesive bond to that of FIG. 4B.

FIG. 5A illustrates a flow chart of a second embodiment of a method of attaching a populated microarray to a package.

FIG. 5B illustrates a cross sectional view of a second embodiment of the packaged microarray apparatus of the present invention that was assembled using the preferred package type illustrated in FIG. 2A and comprises a second embodiment of the adhesive bond.

FIG. 5C illustrates a magnified cross sectional view of an adhesive bond and seal at a microarray/package interface of a packaged microarray apparatus of the present invention using either of the package types of FIGS. 2A or 2C.

FIG. 5D illustrates a cross sectional view of a second embodiment of the packaged microarray apparatus of the present invention that was assembled using the package type illustrated in FIG. 2B and comprises an alternative second embodiment of the adhesive bond to that of FIG. 5B.

FIG. 6A illustrates a flow chart of a third embodiment of a method of attaching a populated microarray to a package.

FIG. 6B illustrates a cross sectional view through a third embodiment of the packaged microarray apparatus of the present invention that was assembled using the package type illustrated in FIG. 2A and comprises a third embodiment of the adhesive bond.

FIG. 6C illustrates a magnified cross sectional view of an adhesive bond and seal at a microarray/package interface of a packaged microarray apparatus using either of the package types illustrated in FIGS. 2A and 2C.

FIG. 6D illustrates a cross sectional view of a third embodiment of the packaged microarray apparatus of the present invention that was assembled using the package type illustrated in FIG. 2B and comprises an alternative third embodiment of the adhesive bond to that of FIG. 6B.

### MODES FOR CARRYING OUT THE INVENTION

### Definitions

The following terms are intended to have the following general meanings as they are used herein:

Nucleic acid—a high molecular weight material that is a polynucleotide or an oligonucleotide of DNA or RNA.

Polynucleotide—a compound or composition that is a polymeric nucleotide or nucleic acid polymer. The polynucleotide may be a natural compound or a synthetic compound. In the context of an assay, the polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. It is thus obvious that isolation of a polynucleotide from the natural state often results in fragmentation. The polynucleotides include nucleic acids, and fragments thereof, from any source in purified or unpurified form including DNA, double-stranded or single-stranded (dsDNA and ssDNA), and RNA, including t-RNA, m-RNA, r-RNA, mitochondrial DNA and RNA, chloroplast DNA and RNA, complementary DNA (cDNA) (a single stranded DNA that is complementary to an RNA and synthesized from the RNA in vitro using reverse transcriptase), DNA/RNA hybrids, or mixtures thereof, genes, chromosomes, plasmids, the genomes of biological materials such as microorganisms, e.g. bacteria, yeasts, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, and the like.

Polynucleotides include analogs of naturally occurring polynucleotides in which one or more nucleotides are modified over naturally occurring nucleotides. Polynucleotides then, include compounds produced synthetically (for example, PNA as described in U.S. Pat. No. 5,948,902 and the references cited therein, all of which are incorporated herein by reference), which can hybridize in a sequence specific manner analogous to that of naturally occurring complementary polynucleotides.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

For purposes of this invention, the polynucleotide, or a cleaved fragment obtained from the polynucleotide, will usually be at least partially denatured or single-stranded or treated to render it denatured or single-stranded. Such treatments are well known in the art and include, for instance, heat or alkali treatment, or enzymatic digestion of one strand. For example, double stranded DNA (dsDNA) can be heated at 90-100° C. for a period of about 1 to 10 minutes to produce denatured material, while RNA produced via transcription from a dsDNA template is already single-stranded.

Oligonucleotide—a polynucleotide, usually single-stranded, usually a synthetic polynucleotide but may be a naturally occurring polynucleotide. The oligonucleotide(s) are usually comprised of a sequence of at least 5 nucleotides, usually, 10 to 100 nucleotides, preferably, 20 to 60 nucleotides.

Various techniques can be employed for preparing an oligonucleotide. Such oligonucleotides can be obtained by biological synthesis or by chemical synthesis. For short sequences (up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of target polynucleotides binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. For longer sequences standard replication methods employed in molecular biology can be used such as the use of M13 for single-stranded DNA as described in J. Messing (1983) Methods Enzymol. 101:20-78.

In situ synthesis of oligonucleotide or polynucleotide probes on a substrate is performed in accordance with well-known chemical processes, such as sequential addition of nucleotide phosphoramidites to surface-linked hydroxyl groups, as described by T. Brown and Dorcas J. S. Brown in Oligonucleotides and Analogues A Practical Approach, F. Eckstein, editor, Oxford University Press, Oxford, pp. 1-24 (1991), and incorporated herein by reference. Indirect synthesis may be performed in accordance biosynthetic techniques (e.g. polymerase chain reaction “PCR”), as described in Sambrook, J. et al., “Molecular Cloning, A Laboratory Manual”, 2^{nd} edition 1989, incorporated herein by this reference.

Other methods of oligonucleotide synthesis include phosphotriester and phosphodiester methods (Narang, et al., (1979) Meth. Enzymol. 68:90) and synthesis on a support (Beaucage, et al. (1981) Tetrahedron Letters 22:1859-1862) as well as phosphoramidate techniques (Caruthers, M. H., et al., “Methods in Enzymology,” Vol. 154, pp. 287-314 (1988) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Aca. Sci. USA (1994) 91:5022-5026.

Protein—a complex high polymer containing chains of amino acids connected by peptide linkages. Proteins are synthesized naturally and synthetically and have functional forms as enzymes, hemoglobin, hormones, viruses, genes, antibodies and nucleic acids. Simple proteins contain only amino acids. Conjugated proteins contain amino acids plus nucleic acids, carbohydrates, lipids, etc. Protein solubility can be classified as the ‘albumin’ class, which is water soluble; the ‘globulin’ class, which is water insoluble but soluble in aqueous salt solution; and the ‘prolamine’ class, which is soluble in alcohol-water mixtures, but not in alcohol or water alone.

Hybridization (hybridizing) and binding—in the context of nucleotide sequences these terms are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

Conventional hybridization solutions and processes for hybridization are described in J. Sambrook, E. F. Fritsch, T. Maniatis, Molecular Cloning: A Laboratory Manual, Cold Spring Harbor Laboratory Press, Cold Spring Harbor, Ed. 2^{nd}, 1989, vol. 1-3, incorporated herein by reference. Conditions for hybridization typically include (1) high ionic strength solution, (2) at a controlled temperature, and (3) in the presence of carrier DNA and surfactants and chelators of divalent cations, all of which are well known in the art.

Typically, hybridizations using synthetic oligonucleotides are usually carried out under conditions that are 5-10° C. below the calculated melting temperature Tₘ of a perfect hybrid to minimize mismatched or non-Watson/Crick base pairing between the probe and target, and maximize the rate at which Watson/Crick base pairs form. The microarray is hybridized for a period of time ranging from about 2 hours to about 2 days, depending on the make-up of the probes (i.e., probe length and diversity of probe composition) and the complexity of the target, for example, at a controlled temperature, which typically ranges from 20° C. to 70° C., depending on the melting temperature Tₘ, as discussed above. Low temperature hybridizations are performed at about 20° C. to about 50° C. (typically about 37-45° C.). High temperature hybridizations are performed at or around 55° C. to about 70° C. (typically 60° C. to 65° C.). However, for most nucleic acid microarrays, high temperature hybridizations are preferred in the art since the higher temperature maximizes the rate of Watson/Crick base pairing of nucleotides, while low temperature hybridizations typically use a co-solvent to lower the Tₘ to maximize Watson/Crick base pairing. The typical time period for hybridization of a microarray is overnight or longer (i.e., anywhere from 8 hours to 24 hours) at the hybridization temperature so as to hybridize the target. The array is then washed and may be dried (if fluorescein is the dye, it scans better wet) and scanned to measure the degree of hybridization using conventional methods and equipment that are well known in the art. The washing step may be performed at around 4° C., however, using temperatures of 0° C. and below are not uncommon.

Moreover, hybridization “completion” is dependent on the user and the target nucleic acid material to be hybridized. Hybridization could comprise anywhere from 1% to 100% of the substrate-bound nucleic acids on at least one feature of the microarray being hybridized with the target material, for example. The hybridization time periods to achieve completion within the 1% to 100% range will vary greatly. The hybridization time periods at the higher temperatures usually are about 8 hours to about 24 hours (or longer) typically, to achieve optimum results or throughput, when the nucleotide make-up of the mixture of probes is diverse and/or the target population is complex.

For protein binding assays see for example, Figeys, Daniel and Devanand Pinto, “Proteomics on a chip: Promising Developments”, Electrophoresis 2001, 22, 208-216—review article; “Rapid protein display profiling of cancer progression directly from human tissue using a protein biochip”, Drug Development Research, 49:34-42 (2000); “Protein Arrays Step out of DNA's Shadow”, Science 289:1673, Sep. 8, 2000—brief news article; and “Protein microarrays for highly parallel detection and quantitation of specific proteins and antibodies in complex solutions”, Genome Biology 2001, 2(2) research 0004.1-00004.13, all of which are incorporated herein by reference in their entirety.

Substrate or surface—a porous or non-porous water insoluble support material. The surface can have any one of a number of shapes, such as strip, plate, disk, rod, particle, including bead, and the like. The substrate can be hydrophobic or hydrophilic or capable of being rendered hydrophobic or hydrophilic and includes inorganic powders such as silica, magnesium sulfate, and alumina; natural polymeric materials, particularly cellulosic materials and materials derived from cellulose, such as fiber-containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. A siliceous substrate is any substrate material largely comprised of silicon dioxide. Natural or synthetic assemblies such as liposomes, phospholipid vesicles, and cells can also be employed.

Common substrates used for microarrays are surface-derivatized glass or silica, or polymer membrane surfaces, as described in Z. Guo et al. (cited above) and U. Maskos, E. M. Southern, Nucleic Acids Res 20, 1679-84 (1992) and E. M. Southern et al., Nucleic Acids Res 22, 1368-73 (1994), both incorporated herein by reference. In modifying siliceous or metal oxide surfaces, one technique that has been used is derivatization with bifunctional silanes, i. e., silanes having a first functional group enabling covalent binding to the surface (often an Si-halogen or Si-alkoxy group, as in SiCl₃ or —Si(OCH₃)₃, respectively) and a second functional group that can impart the desired chemical and/or physical modifications to the surface to covalently or non-covalently attach ligands and/or the polymers or monomers for the biological probe array. See, for example, U.S. Pat. No. 5,624,711 to Sundberg, U.S. Pat. No. 5,266,222 to Willis and U.S. Pat. No. 5,137,765 to Farnsworth, each incorporated herein by reference.

Adsorbed polymer surfaces are used on siliceous substrates for attaching nucleic acids, for example cDNA, to the substrate surface. Substrates can be purchased with a polymer coating, or substrates can be coated with a solution containing a polymer and dried according to well-known procedures. For a typical protocol for substrate coating see website http://cmgm.stanford.edu/pbrown/protocols/1_slides.html.

Immobilization of oligonucleotides on a substrate or surface may be accomplished by well-known techniques, commonly available in the literature. See, for example, A. C. Pease, et al., Proc. Nat. Acad. Sci. USA, 91:5022-5026 (1994); Z. Guo, R. A. Guilfoyle, A. J. Thiel, R. Wang, L. M. Smith, Nucleic Acids Res 22, 5456-65 (1994); and M. Schena, D. Shalon, R. W. Davis, P. O. Brown, Science, 270, 467-70 (1995), each incorporated herein by reference.

Feature—a feature is a biological material bonded to an array substrate in a spatial arrangement of locations. The location of a feature is spatially addressable, typically by a row and column location, for example. An array or wafer comprises a plurality of sets of features that can be divided up into microarrays of individual sets or multiple sets of features.

### DETAILED DESCRIPTION OF THE INVENTION

The present invention is directed to a method of bonding a microarray die to a microarray package such that the bond between the die and the package is stable and reliable under thermal stress. Further, the invention is directed to a packaged microarray apparatus comprising a package, a microarray and an adhesive bond between the microarray and package that is stable and reliable under thermal stress.

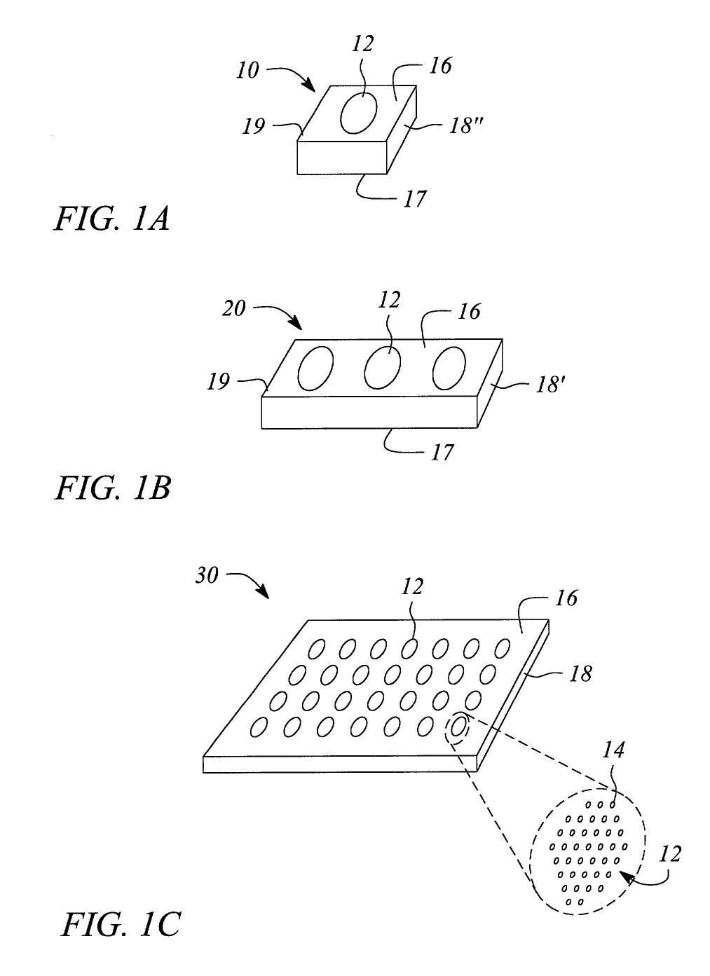

For purposes of the invention, a microarray 12 is defined as having a plurality of biological features 14 spatially arranged on a surface 16 of a substrate 18 so that the features 14 are addressable on the substrate 18. FIG. 1A illustrates a microarray die 10 comprising the microarray 12 on a small substrate 18″ and FIG. 1B illustrates a microarray die 20 comprising multiple microarrays 12 on a relatively larger substrate 18′. The substrates 18′ and 18″ are incrementally smaller portions of an array substrate 18. The substrates 18, 18′, 18″ are defined as a substrate that supports the biological features 14 on its surface 16. The biological features 14 are illustrated in a magnified view in FIG. 1C. For convenience of manufacture, it is often advantageous to make a plurality of microarrays 12 on the array substrate 18 (or ‘wafer 30’), as illustrated in FIG. 1C. In some embodiments, after the microarrays 12 of features are formed, the array substrate 18 is cut, diced or separated into the individual die 10 or 20 containing smaller numbers of microarrays 12, such as that shown in FIGS. 1A and 1B respectively. In other embodiments, the microarray 12 is populated with biological features after the array substrate 18 is cut or diced into the individual die 10, 20 or substrates 18′, 18″. Depending on the embodiment, the microarray 12 may be formed before or after the microarray substrate 18′, 18″ is bonded to a package. The terms ‘microarray’, ‘microarray die’ and ‘microarray substrate’ may be used interchangeably, unless otherwise specified herein. Moreover as used herein, the term ‘microarray’ does not imply that biological features have been attached, unless otherwise specified.

In some embodiments, microarrays 12 are built on substrates 18 that are cut into 1″×3″ (25 mm×75 mm) glass slides, for example, as illustrated for microarray die 20 in FIG. 1B. Forty-eight slides can be manufactured from one 320×340 mm substrate 18, for example. In other embodiments, 196 microarray die 10, as illustrated in FIG. 1A, sized at 22×22 mm, for example, can be obtained from the same array substrate 18. Preferably, the smaller sized microarray die 10 is bonded to a package to facilitate handling and processing, such as assaying the biological features 14 of the microarray 12.

The substrates 18, 18′, 18″ are illustrated as rectangular or square in FIGS. 1A-C, however it is within the scope of the invention for the substrates to have other shapes, ranging from circular to square and rectangular to elliptical, for example. It is not the intent of the invention to be limited by a particular substrate shape or material. Moreover, the sizes of the substrates 18, 18′, 18″ are not to scale in FIGS. 1A, 1B and 1C. Further, the number of microarrays 12 illustrated, as well as the number of features 14 illustrated in a microarray 12, in FIGS. 1A-1C are illustrative only and not intended to limit the scope of the invention. Moreover, typical substrate 18 materials used for molecular array applications according to the invention are soda lime glass, pure silica, heat resistant glasses, or other siliceous materials (hereinafter collectively referred to as ‘glass’) that are optically transparent. It is not the intent of the invention to be limited to these materials. Other common substrate materials are known in the art and are within the scope of the present invention.

The invention is directed to in situ synthesized nucleic acid arrays on array substrates using conventional in situ synthesis methods well known in the art and to nucleic acid arrays or protein arrays that are created by depositing material synthesized naturally or synthetically (i.e., presynthesized). In situ synthesized microarrays of nucleic acids are expensive to manufacture due to the repetitive nature of the in situ synthesis process and the volumes of chemicals required at each step of the synthesis. It is desirable to manufacture the highest quantity of arrays on the substrate or wafer as possible to minimize cost. The volume of chemicals used in the process is proportional to the size of the substrate and not to the number of arrays manufactured. Therefore, it is advantageous to maximize usage of the substrate surface for arrays and minimize the usage of the substrate surface for such things as array identification and handling.

For example, arrays synthesized in a 1″×3″ slide format (25 mm×75 mm) typically reserve 10 to 15 mm of the slide for a bar code label or other identification means. It is necessary to provide a unique identification means to each substrate and preferable to have a unique identification means for each array so that the biological features on the array can be linked to an original design. Otherwise, other means to identify the feature's identity after the hybridization would be needed. The slides generally are not permanently attached to any other materials for processing. As such, this area left for identification does not contain an array, yet it is washed by all of the chemicals required for each in situ synthesis cycle. For the purposes of the invention, the microarray die 10, 20 that are cut from a wafer 30 are packaged for handling and processing.

A microarray package that supports individual microarray die 10, 20 is a lower cost material that can provide space for array identification and for handling. Therefore, a higher proportion of the substrate surface is made available to use for more arrays. By way of example, a bar code can cover an area that is approximately 10 mm×20 mm. The spotting density of in situ synthesis equipment provides a new biological feature on 200 micron centers. Therefore, the bar code covers an area equivalent to an additional 5000 biological features, such that the package essentially allows for 5000 more features to be added to microarrays, thereby saving on in situ processing cost. Other advantages of using a microarray package are discussed further below. A microarray package serves multiple functions in addition to providing lower cost space for the array identification means. For example, a microarray package functions to orient the microarray die correctly for scanning. A microarray of biological features on a glass substrate is not visible to the unaided eye and a square shaped glass die, for example, can be placed into a scanner in eight possible orientations. Prepackaging the microarray into a package properly orients and locates the microarray in the scanner for proper scanning and subsequent data extraction and probe identification.

Moreover, housing a microarray die in a package provides a second function of protecting the microarray from damage and debris. The substrate material can be easily broken or chipped in handling. Bonding the microarray die to a package provides a protective rim around the periphery of the die and minimizes chipping or breakage. The microarray package comprises a frame that surrounds an interior opening. The microarray is bonded to the frame. Further, when the package further comprises or accepts a lid or cover, the biological features on the microarray can be enclosed within the package. The package will prevent fingerprints, dust and other debris from contacting the microarray surface. Dust and other debris interfere with optical scanning results. When the microarray is scanned by sensitive fluorescent scanners, dust and debris will be detected as unwanted background signal and may degrade the quality of the data from the array. Fingerprints and debris are also a source of RNase, an enzyme that destroys mRNA. If the microarray is used in an expression profiling assay designed to detect and quantify the amounts of specific messenger RNA, any RNase contamination is a serious problem as it will destroy the mRNA before it can hybridize to the microarray.

Certain packages also can provide a third function of providing all of the fluidic control necessary for a hybridization. A package that provides fluidic control for a hybridization assay is described and illustrated in U.S. Pat. No. 6,261,523, issued Jul. 17, 2001 entitled “Adjustable Volume Sealed Chemical-Solution-Confinement Vessel”, which is incorporated by reference herein in its entirety. Since each microarray die is bonded into its own package, the sample and reagents for a specific assay can be introduced into the fluidic control package in minute quantities and be subsequently rinsed therefrom. A packaged microarray can distribute the sample across the microarray surface and minimize sample evaporation during a hybridization assay. For example, microarrays for expression profiling assays must be placed in contact with a sample for extended times, typically 1 to 24 hours, for hybridization. Typically the hybridization is conducted at elevated temperatures such as 37-65° C. The sample is precious and normally available in very small quantities. Therefore, only a very thin layer of sample fluid covers the microarray surface. This thin layer is subject to evaporation, unless protected therefrom. Packaged arrays conserve this sample. Manual methods for hybridization include distributing the sample with a cover slip over the microarray surface and placing the assembled microarray and cover slip in a secondary containment system to prevent evaporation. However, it is more convenient to use a packaged microarray that ensures that the sample is completely distributed across the microarray surface and will not evaporate during the hybridization

Therefore, a microarray package is defined as a housing comprising a frame that supports a microarray die 10, 20. The package or its frame orients the die substrate 18″, 18′ correctly for scanning, and protects the microarray from damage, contamination and debris. In one or more embodiments, the package also provides for a cover, lid or other enclosure. In some of those embodiments, the closable package may support all of the fluidic control necessary for assays. For simplicity of the discussion herein, the terms ‘package’ and ‘frame’ are used interchangeably herein, unless otherwise specified.

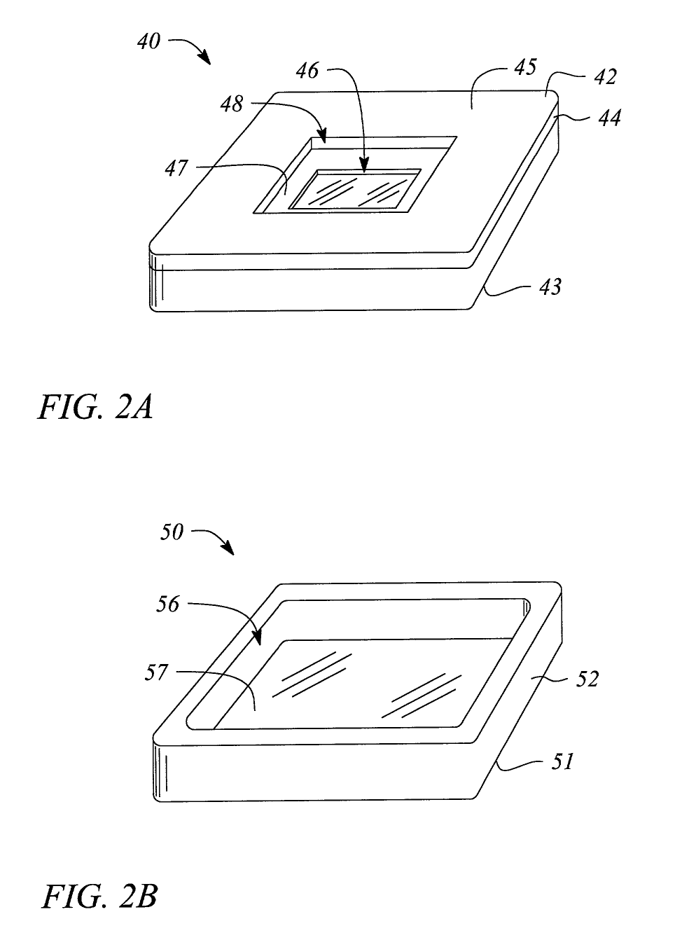

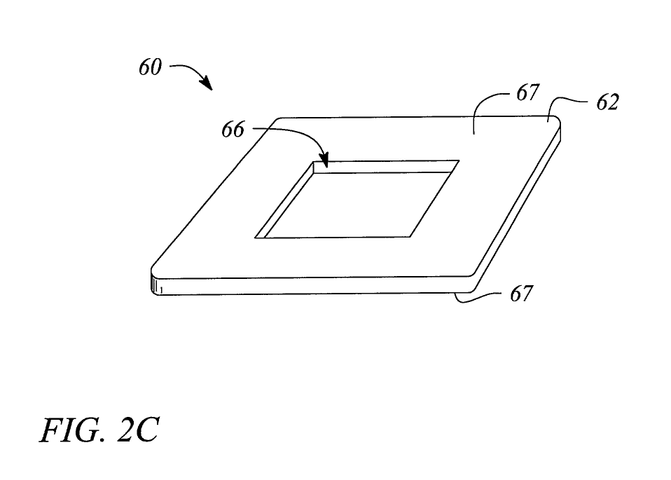

The present invention is directed to using different types of packages. One type of package is illustrated generally in FIG. 2A. The package 40 has a cover 43 that encloses a frame 42 on one side. The frame 42 has an interior opening 46 framed by a recess 48 in an opposite side or outside surface 45 of the frame 42. The opening 46 is slightly smaller than the size of a microarray die 10, 20, but the recess 48 is slightly larger than the microarray die size and at least as deep as the thickness of the die substrate 18′, 18″. The microarray 10, 20 is placed into the recess 48 in the frame 42 with the populated array surface 16 facing the package interior. A perimeter 19 of the array surface 16 is bonded to a ledge surface 47 of the recess 48 that surrounds the opening 46, such that the bond provides a fluid tight seal at peripheral ledge surface 47 of the opening 46 and the opening 46 essentially frames the microarray 12 on an interior of the package 40. The microarray 12 is exposed to the package interior and is accessible for interacting with a sample and reagents for an assay within the package 40 that may be introduced into the package a variety of ways. Either or both of the sample and the reagents are fluids for the purposes of the discussion herein. A fluid tight seal is one that substantially delays, or otherwise effectively prevents, the escape or evaporation of a fluid, especially during the time period of an assay.

U.S. Pat. No. 6,261,523 describes specific embodiments of the package type 40 and the introduction of fluids therein to a microarray, which are incorporated herein by reference. The specific embodiments will not be described herein. Alternatively, the lid or cover 43 can be removed to expose the interior of the package 40 and to introduce fluids that contact with the array surface 16 for an assay. Where the cover 43 is removed to add fluids, the frame 42, or an assembly welded to the frame, may further have a perpendicular sidewall 44 around a periphery of the frame 42, such that the frame 42 is essentially a well that can accommodate fluids. The sidewall 44 receives or mates with the lid or cover 43. A surface 17 of the microarray 10, 20 that is opposite the array surface 16 is recessed into, or can be flush with, the outside surface 45 of the frame 42 after assembly. Also, it is within the scope of the invention for the surface 17 of the microarray 10, 20 to extend or protrude from the outside surface 45 of the frame 42. Scanning the microarray 12 is achieved through the opposite surface 17 or the cover 43 is removed for scanning.

While the present invention is described herein with respect to a preferred package type 40 and variations thereof, the present invention is also applicable to attaching a microarray die 10, 20 to a type of package that is illustrated in FIG. 2B. The package 50 has a solid bottom 51 and a frame 52 having perpendicular sidewalls creating a well or cavity having an interior opening 56. The opposite surface 17 of the microarray die is bonded or attached to an interior surface 57 of the bottom 51. The array surface 16 comprising the biological features 14 faces the opening 56 in the package 50 that is opposite to the interior surface 57. The frame 52 receives a lid (not shown) over the opening 56 to enclose the microarray die 10, 20.

In another embodiment illustrated in FIG. 2C, a package 60 comprises a frame 62 having an interior opening 66 similar to the package 50, but no bottom 51 or interior attachment surface 57, as in the package 50. In essence, the package 60 also resembles the frame 42 without the recess 48 of package 40. The array surface 16 comprising the biological features 14 is attachable to either outer attachment surfaces 67 of the frame 62 with the features 14 facing into the opening 66. The frame 62 has a thickness dimension, or perpendicularly extending sidewalls, to provide depth to the opening 66. In some embodiments, the microarray 12 can be sandwiched between two frames 62, or two frame portions having relatively thinner thickness dimensions, in a way similar to the way a 35 mm photographic slide is framed. In some of the embodiments, the package 60 further comprises a lid (not shown) that attaches to the attachment surface 67 opposite to the attachment surface to which the microarray 12 is attached.

For either package type 50 or 60, the microarray 12 is accessible for interacting with a sample and reagents for an assay within the package 50, 60 that are introduced into the package 50, 60 via the opening 56, 66. The opening 56, 66 can be covered with a removable lid. In one embodiment, the lid can be a re-sealable, flexible, pliable film, such as plastic or metal foil film, which is attached to the frame 52 62, and covers the opening 56, 66. The pliable film can be peeled back to introduce fluids for an assay and re-attached. The microarray 12 is scanned when the package lid is removed or the lid is made from a material that is transparent to the type of scan.

However, the bonding of the microarray die 10, 20 to a package presents some challenges. One challenge that the present invention addresses is a thermal mismatch between the material of the microarray substrate and the material of the package. The array is typically made from glass, which has a thermal expansion rate of approximately 3.2×10⁻⁶/° C. The package is made of plastic, typically an injection molded plastic. Most plastics, which are convenient for injection molding the package, have coefficients of thermal expansion (CTE) that are an order of magnitude higher than the CTE rate of glass. The thermal mismatch between the materials is an important consideration where the packaged microarray will be exposed to high and low temperature extremes. More particularly, the thermal mismatch of the different materials is important where the package types 40, 60 are used, since an intact fluid tight seal between the microarray and the package over the interior opening 46, 66 is desirable before and during temperature extremes of an assay.

A typical packaged microarray might be exposed to uncontrolled high and low temperature extremes for unpredictable periods of time during shipping and handling of the packaged microarray before it is used by a user in an assay. Further, a typical packaged microarray is exposed to controlled high and low temperature extremes for specified periods of time during an assay. The uncontrolled temperature extremes can range from a high temperature of at least about 70° C. to a low temperature of less than about −30° C. for unpredictable periods of time associated with shipping the packaged microarray to different parts of the world at different times of the year. However, the temperature extremes associated with shipping a packaged microarray typically are imposed gradually on the packaged microarray. The controlled temperature extremes of an assay can range from a high temperature of at least about 70° C. to a low temperature of less than about 0° C. for different, albeit specified or known, periods of time. However, the temperature extremes associated with an assay typically are imposed relatively abruptly on the packaged microarray. Hybridization assays are described in more detail elsewhere in this application (see for example, the Definitions section and the Examples section). The temperature extremes mentioned herein are exemplary and not intended to limit the scope of the present invention. For the purposes of the invention, the adhesive bond between the microarray and the package of the packaged microarray preferably is intact before an assay and remains intact at the completion of the assay. By ‘intact’ it is meant that the bond has not cracked, such that the microarray is separated from the package, either partially or completely, and with respect to package types 40 and 60, the term ‘intact’ further means that the bond remains fluid tight.

For example, polypropylene is a highly desirable choice for these plastic packages due to its low cost, ease of manufacturing, high working temperature limit and its passive surface that minimizes the risk that a DNA or a RNA sample will stick to the package surface instead of binding to the microarray 12. The coefficient of thermal expansion of polypropylene is 50×10⁻⁶/° C. ABS plastic is another desirable choice for the plastic package of the present invention. ABS plastic has a coefficient of thermal expansion of about 40×10⁻⁶/° C.

The present invention attempts to account for the thermal mismatch between the materials by making the bond between the microarray and the package sufficiently strong and flexible to withstand the difference in thermal expansion between the glass and the plastic during temperature extremes, such as those described above. For example, if the bond fails before or while using the packages 40 and 60 in an assay, a crack may open a seal between the microarray and the package along the ledge surface 47 surrounding the opening 46 or along the attachment surface 67 surrounding the opening 66, respectively, thereby allowing a sample solution introduced into the package 40, 60 for an assay to evaporate or escape. Premature loss of the sample will likely ruin the assay.

Another challenge that the present invention addresses is a relative reluctance of some adhesives to wet to some microarray substrates and/or to some packages for bonding. For example, the surface of a microarray substrate typically is derivatized for chemical bonding to the surface during in situ synthesis of biological features, and also during immobilization of presynthesized biological features. For in situ synthesis of biological features, the first monomers of the feature sequences are attached to the substrate surface that has been derivatized with silane-containing compounds, or other compounds, known in the art to facilitate the bonding of the first monomers in in situ synthesis. Subsequent monomers are added directly to the monomers of the growing feature chain. For deposition of presynthesized features, such as cDNA or protein probes or targets, the presynthesized feature is attached to a polymer adsorbed or coated on the surface of the substrate to facilitate bonding. The adsorbed polymer is coated and dried on the substrate surface. However, the derivatizations described above typically render the substrate surface hydrophobic or otherwise difficult for adhesives to wet to.

Further, the surface of the plastic housing may be hydrophobic or otherwise has a surface energy incompatible with adhesives. Microarray packages typically are made from injection molded plastic. Polypropylene and ABS plastic, for example, are the most common injection molded package materials. Polypropylene has a very low surface energy rendering it hydrophobic. Moreover, the surfaces of injection molded plastics may have surface contaminants as a result of the injection molding process or subsequent handling that render them difficult to bond with adhesives. Adhesives conventionally tend to bond poorly to hydrophobic surfaces since they cannot wet to these surfaces. The surface energy of the material to be bonded desirably has a higher surface energy than the surface energy of the adhesive used for bonding to the material in order for the adhesive to wet or stick to the surface of the material. Conventional chemicals typically employed to modify the surface energy of plastics often interfere with the hybridization process and thus, are not recommended for the present invention.

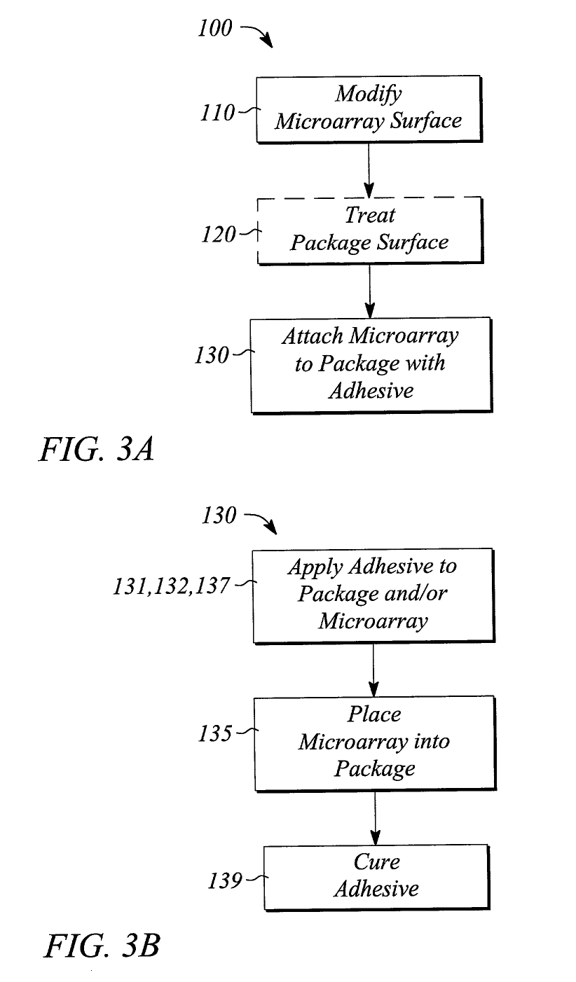

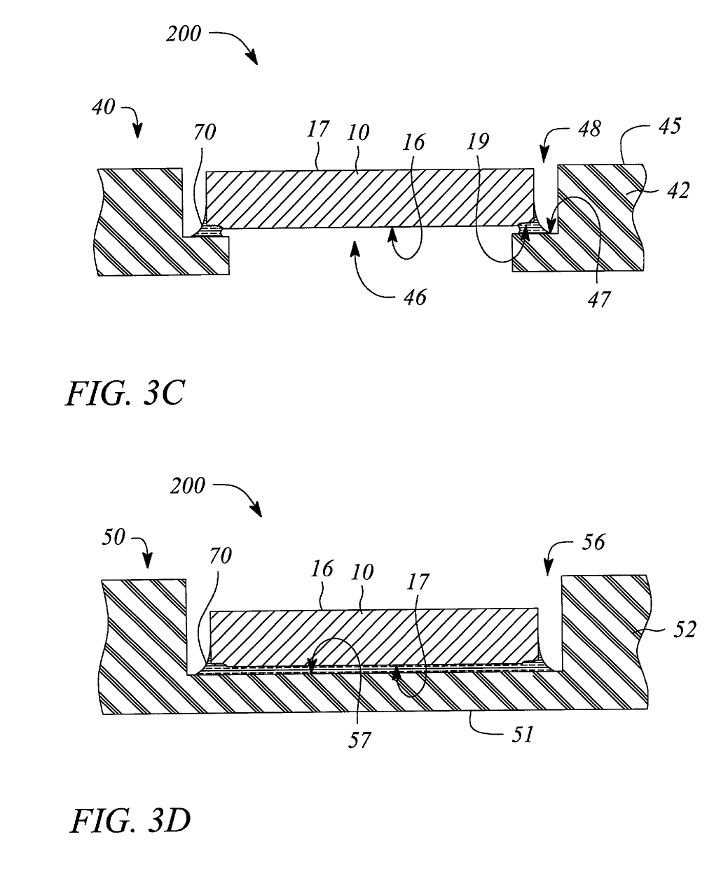

In accordance with one aspect of the present invention, a method of bonding a microarray to a microarray package is provided. FIG. 3A illustrates a flow chart of a method 100 of bonding according to the invention. The method 100 comprises modifying 110 a bonding surface of the microarray to render the bonding surface more wettable by an adhesive. For the purposes of the method 100, the microarray is defined above either as having a plurality of biological features populated on an array surface of an array substrate, or as a microarray substrate before it is populated with features. Therefore, in some embodiments, the microarray already has biological features before the step of modifying 110 a bonding surface thereof. In other embodiments, the microarray substrate is modified prior to populating a surface with biological features. The method 100 further comprises attaching 130 the microarray to the package using the adhesive between the modified bonding surface and an attachment surface of the package. In some embodiments, the method 100 optionally further comprises treating 120 the attachment surface of the microarray package where the microarray is to be attached to render the attachment surface more wettable by the adhesive. The modified and treated surfaces are rendered ‘more wettable’ by the adhesive than the wettability of the respective surfaces by the adhesive before the respective surface treatments.

In a preferred embodiment, the step of modifying 110 the bonding surface of the microarray comprises simultaneously dicing an array substrate or wafer comprising a plurality of microarrays into individual microarray die, and grinding the bonding surface at a perimeter of each individual microarray die. The bonding surface is ground to remove a very thin surface layer from the perimeter. Simultaneously dicing and grinding is preferred for the step of modifying 110 because it is faster and more convenient to dice and grind in a single pass. In this preferred embodiment, the bonding surface is the array surface 16 that ultimately comprises the biological features 14. The perimeter 19 is outside of and surrounds an attachment area of the array surface 16 for the biological features 14. In other embodiments, the bonding surface is the opposite surface 17 of the microarray.

A Disco dicing system having two blades, model no. DFD651 manufactured by Disco Corp. of Tokyo, Japan, was used in the step of modifying 110 according to the preferred embodiment. A first blade grinds a thin surface layer from the perimeter 19 of each die. This blade removes the hydrophobic surface layer from the array substrate without damage to the microarray. In a more preferred embodiment, the first blade was a 1.5 mm wide blade. A second blade cuts through the glass array substrate to separate each microarray into individual die. In a more preferred embodiment, the second blade was a 300 micron wide blade. The second blade is centered relative to the first grinding blade. Other systems with dual blade designs that are useful for this step of modifying 110 of the method 100 are manufactured by Manufacturing Technologies (MTI) of Ventura, Calif. (www.mtionline.com) and Kulicke and Soffa (K&S) of Willow Grove, Pa. (www.kns.com).

In an alternative embodiment, the dicing and grinding in the step of modifying 110 may be accomplished separately. However, this alternative embodiment is less desirable, since performing the dicing and grinding processes separately requires additional time and handling of the fragile microarrays. Nevertheless, this alternative embodiment is within the scope of the present method 100.

In yet another alternative embodiment, the step of modifying 110 transforms the bonding surface, which is hydrophobic and has a low surface energy, to a relatively higher surface energy surface. In this alternative embodiment, the bonding surface preferably is on the array surface side. The step of modifying 110 comprises masking the array surface of the microarray, such that the mask protects the area for biological features attachment and the perimeter is exposed, and applying an electrical discharge or corona to the exposed perimeter surface. Equipment, such as the Tantec Spot Treater Electrical Surface Treatment System (Schaumburg, Ill.) or other plasma treating systems may be used to transform the surface energy of the bonding surface, as long as the microarray is masked to prevent damage to the fragile biological features, or the array surface to which the biological features are ultimately attached.

In other embodiments, the step of modifying 110 comprises masking off the bonding surface before the array surface is treated or derivatized for the attachment of the biological features. In these embodiments, the derivatization chemicals that render the surface hydrophobic, as disclosed above, do not contact or alter the surface energy of the masked bonding surface. The mask is removed prior to the step of attaching 130 the microarray to the package using the adhesive.

Alternatively, if the microarray is attached 130 to the package prior to populating the array surface with biological features, the surface derivatization step, which renders the surface hydrophobic for feature attachment, is not performed until after the microarray and package are attached together. Therefore, the bonding surface is not modified 110. Where the bonding surface is the perimeter of the array surface, the package effectively functions as a mask defining the attachment area on the array surface for the chemical derivatization. Therefore, the present invention further provides a method of creating a packaged microarray that comprises masking a portion of a surface of a microarray; and exposing an unmasked portion of the surface to treatment suitable for bonding biological features to the treated unmasked portion, wherein the masked portion is masked by attaching the portion to be masked to an attachment surface of a microarray package. The present invention also provides a method comprising treating a part of a surface of a substrate to bond biological features to the treated part; and providing an untreated part of the surface. Such a method includes attaching the untreated part to a package, such that the package masks the untreated part from the treatment.

Some injection molded plastic materials, including but not limited to polypropylene, have a hydrophobic surface or a surface energy that is lower than what is readily wettable by adhesives. However, the attachment surface of an ABS plastic package, for example, may have sufficient surface energy to be readily wettable by adhesives. Therefore, the step of treating 120 the attachment surface of the package is considered optional for the present invention and its use depends on the package material chosen for the package. The step of treating the package surface 120 is illustrated as a dashed-line box in FIG. 3A for that reason. The optional step of treating 120 an attachment surface of a microarray package advantageously treats the hydrophobic or low surface energy attachment surface of plastic packages to improve their wettability by adhesives. The attachment surface of the microarray package is optionally treated 120, as necessary, to prepare the surface for adhesive bonding.

The step of treating 120 comprises using any one or more of electrical discharge systems, plasma etching and fluorine surface treatment to modify a surface layer of the attachment surface. Other processes known in the art of surface treatment, including but not limited to cleaning, roughening, chemical etching, application of primers, thermal treatment and the like (See Handbook of Plastics Joining, A Practical Guide from the Plastics Design Library), may also be used and still be within the scope of the present invention, as long as they are compatible with subsequent assays of a biological material inside the plastic package.

Electrical discharge systems are open-air corona systems that modify a surface by ionizing gas particles in an air or a nitrogen atmosphere, which subsequently react with the surface of the plastic to roughen it and introduce reactive groups into the surface layer. The reactive groups and the roughening render the surface layer more wettable by an adhesive. Typical discharge systems, such as the Spot Generator HP-S manufactured by Tantec of Schaumberg, Ill. and the high frequency arc system, Tantec EST system, also manufactured by Tantec of Schaumberg, Ill., will work in the step of treating 120 of the present method 100. One skilled in the art can readily determine the parameters for modifying a surface with an electrical discharge system in accordance with the present method 100 without undue experimentation with the information disclosed herein and that from the system manufacturer.

Plasma etching is a well-known technique for removing thin surface layers using high energy ions in the form of plasma to bombard the surface and displace surface layer moieties, for example. Many gases can be used in the plasma chamber during the plasma process including, but not limited to normal atmosphere, N₂, O₂, CF₄, Argon, etc. The process parameters that can be varied in the plasma process include pressure, flow rate of gas, chamber temperature, RF power, and process time. One skilled in the art can readily determine the parameters for etching a surface layer with a plasma etching system without undue experimentation with the information provided herein and that from the plasma system manufacturer. A typical plasma etcher that is useful for the step of treating 120 of the present method 100 is model no. 7100, manufactured by Metroline/IPC of Corona, Calif. Preferred parameters for the plasma process included using O₂ gas, 0.55 Torr pressure, a flow rate of 500 SCCM (standard cubic centimeters per minute), a temperature range of 45-150° C., 500-800 W RF power, and a process time of 5-8 minutes using Plasma etcher Model No. 7100 from Metroline/IPC of Corona, Calif.

Fluorine surface treatment is a permanent surface modification treatment. Through a gas-surface reaction with fluorine gas, a thin fluorocarbon barrier layer can be created on surfaces of polyolefin articles. Fluorination treatment is a permanent molecular bonding of fluorine atoms to exposed surfaces of the material to be treated. Fluorination treatment improves the wettability of the surface of the substrate being treated through a combination of destruction of oils and waxes on the polymer surface and the creation of polar groups on polymer surface. The result is an increase in surface energy similar to that attained by flame or corona surface treatments. This facilitates proper wet out by adhesives and allows bonding to occur.

However, there is one important difference between surface modified fluorination and other surface modifications, such as those described above. While the effects of flame and corona treatments are known to fade quickly with time, surface modified fluorination treatments are very long lived and have no reported lifetime limits. With surface modified fluorination, a second effect is operative as well; surface gloss is reduced by the surface modified fluorination reaction. The resultant microtexture of surface modified fluorinated polymer surface is clearly visible in a scanning electron microscope and it is believed that this micro-roughened texture at the surface permits greater bond strengths to be achieved with most inks and adhesives. This roughening phenomenon is not known to occur with the other more common surface treatment processes, which only alter surface energy. Fluoro-Seal, Inc. of Houston, Tex. is one example of a company that provides surface fluorination treatment. Fluoro-Seal, Inc. of Houston, Tex. performed fluorine processes on test samples provided by the inventors. Both fluoro seal level 1 and fluoro prep surface treatments were run at Fluoro-seal's Allentown, Pa facility on the test samples.

The electrical discharge surface treatment was preferred for the step of treating 120 of the present method 100, primarily due to convenience. The electrical discharge treatment process is serial instead of batch and very fast. The surface treatments were comparable between the processes, both ‘batch to batch’ using the electrical discharge process, and between the different electrical discharge, plasma etch and fluorine treatment processes.

The step of attaching 130 is illustrated in FIG. 3B. The step of attaching 130 the microarray comprises applying 131, 132, 137 an adhesive to one or both of the package or the microarray, such that the adhesive wicks between the modified bonding surface and the attachment surface when the microarray is placed in contact with the package. For packages 40, 60 the attachment surface is surface 47, 67 that frames the opening 46, 66 and the modified bonding surface of the microarray is the perimeter 19 on the array surface 16. For package 50, the treated attachment surface is the interior surface 57 of the package 50 and the modified bonding surface of the microarray is the opposite surface 17 and preferably, a perimeter portion of the opposite surface 17.

The step of attaching 130 further comprises placing 135 the microarray into the package until the microarray and package make contact with each other or with the adhesive, and the adhesive wicks around a periphery of the microarray die at an interface between the modified bonding surface and the attachment surface; and curing 139 the adhesive. In the step of attaching 130, the step of placing 135 can be performed before or after the step of applying 131, 132, 137. Further, the step of curing 139 can be performed after each application 131, 132, 137 of adhesive.

FIGS. 3C and 3D illustrate cross sectional views of a packaged microarray apparatus 200 according to present invention. In FIG. 3C, the packaged microarray apparatus 200 comprises a microarray die 10, 20 populated with biological features (not illustrated) on the array surface 16 and having a modified bonding surface perimeter 19 that was modified in accordance with the method 100. The packaged microarray apparatus 200 further comprises a package 40 having a frame 42 with an attachment surface 47, that may be optionally treated according to the method 100 also, and a cured adhesive bond 70 that bonds together the microarray 10 and frame 42 at an interface between the modified bonding surface 19 and the attachment surface 47. Only package 40 is illustrated in FIG. 3C with a recess 48 surrounding the attachment surface 47 and opening 46. However, FIG. 3C is applicable to the package type 60 if attachment surface was not recessed, since the adhesive bond 70 is the same for the package 60. For the preferred package types 40, 60, the adhesive bond 70 also functions as a fluid tight seal between the microarray 10, 20 and the frame 42, 62.

In FIG. 3D, the packaged microarray apparatus 200 according to the present invention comprises the microarray die 10, 20 populated with biological features on the array surface 16 and having a modified bonding surface 17, opposite to the array surface 16, that was modified according to the method 100. The packaged microarray apparatus 200 further comprises a package 50 having an attachment surface 57, that may or may not be treated according to the method 100, and a cured adhesive bond 70 that bonds together the microarray die 10, 20 and the package 50 at the modified bonding surface 17 and the attachment surface 57. In some embodiments not illustrated in FIG. 3D, the adhesive bond 70 may extend between the microarray 10, 20 and the package 50 only along the perimeter portion of the modified bonding surface 17. By limiting the adhesive bond 70 to the perimeter portion, noise-related signals that may occur during a subsequent scan of the microarray surface can be reduced.

In another aspect of the method of the present invention, a method of attaching a microarray of biological features to a microarray package is provided. The method comprises using an adhesive system between a bonding surface of the microarray and an attachment surface of the package. The adhesive system is a plurality of separately applied and cured adhesives used in combination to bond the microarray to the package. The system of adhesives provides a more intact bond between the microarray and the package after an assay relative to any one of the system adhesives used individually to bond the microarray and the package. The adhesive system has bond strength and flexibility that are both sufficient enough to keep the bond intact with physical stresses caused by different thermal expansion rates of the microarray and the package when exposed to temperature extremes, such as the temperature extremes of an assay or of shipping, as described above. In this aspect of the method, the microarray bonding surface and/or the package attachment surface may or may not be treated or modified as described above for the method 100, depending on the embodiment.

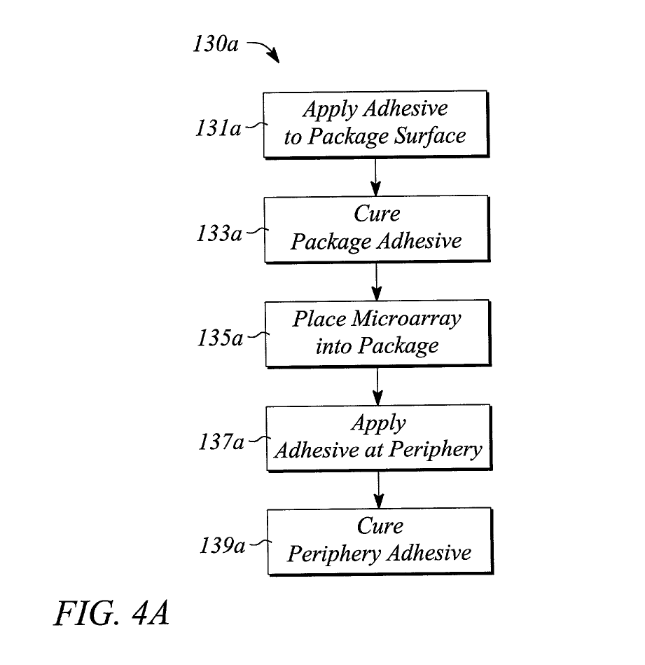

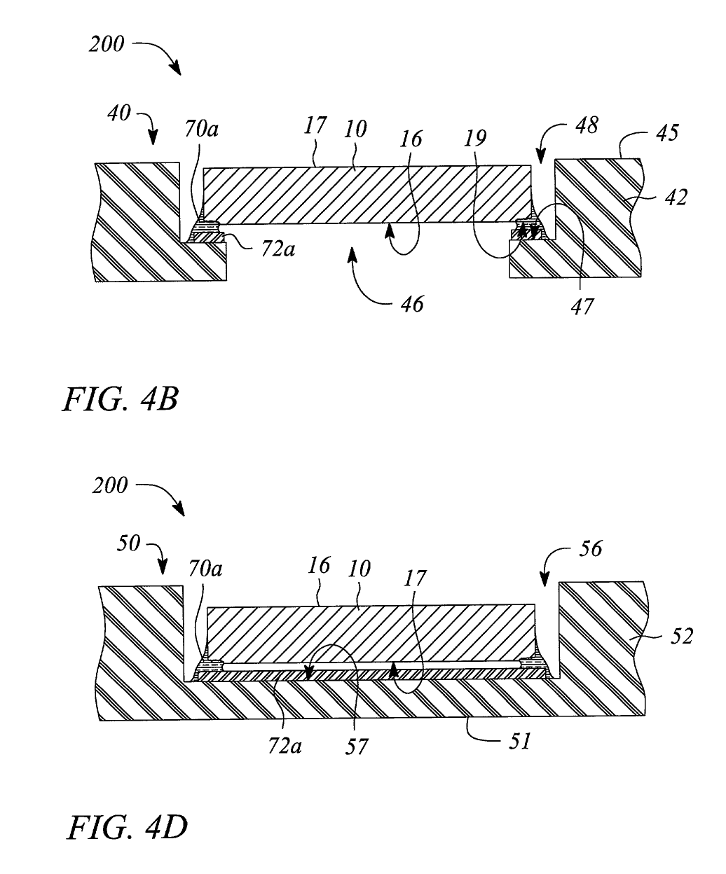

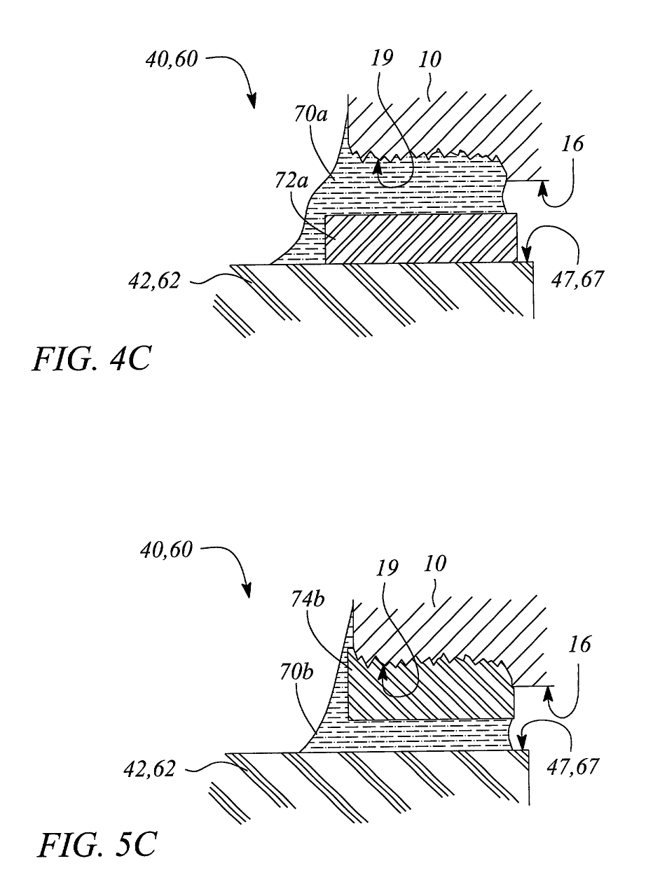

According to the present invention, the method of attaching, and therefore the resultant adhesive bond on the packaged microarray apparatus 200, may be realized in different ways. A first or preferred embodiment of the method of attaching 130a is illustrated in FIG. 4A. The method of attaching 130a comprises applying 131a a first or ‘package’ adhesive to the attachment surface of the package; and curing 133a the package adhesive into a cured package adhesive layer 72a bonded to the package. Preferably the adhesive layer is thin and uniformly spread over the attachment area.

The method of attaching 130a further comprises placing 135a the microarray into the package, such that the bonding surface makes contact with the cured package adhesive layer. The method of attaching 130a still further comprises applying 137a a second or ‘periphery’ adhesive to the periphery of the microarray die adjacent the cured package adhesive layer, such that the periphery adhesive wicks around the entire periphery of the microarray die and into an interface between the microarray die and the cured package adhesive layer on the package along the bonding surface. The method of attaching 130a then further comprises curing 139a the periphery adhesive to form a periphery bond 70a.

The package adhesive adheres well to the package surface, including package surfaces that are optionally treated, as described above for the method 100. The periphery adhesive is much more flexible than the package adhesive and adheres to other cured adhesives and to the bonding and attachment surfaces. According to the first embodiment, the microarray bonding surface and the package attachment surface each may be optionally modified or treated as described above for the method 100.

FIGS. 4B and 4D illustrate cross sectional views of the packaged microarray apparatus 200 that has the adhesive system bond 72a, 70a according to the first embodiment of the method of attaching 130a. FIG. 4B illustrates the apparatus 200 using the preferred package type 40, while FIG. 4D illustrates the apparatus 200 using the package type 50. FIG. 4B also is exemplary of the adhesive system bond 72a, 70a between a microarray die 10, 20 and the frame 62 of package 60 on non-recessed attachment surface 67. Therefore, the packaged microarray apparatus 200 of the first embodiment comprises the microarray die 10, 20, the package 40, 60, 50, and an adhesive system bond 72a, 70a.

In FIG. 4B, the adhesive system bond 72a, 70a bonds the microarray die 10, 20 to the attachment surface 47 of the package frame 42 of the packages 40, at the perimeter bonding surface 19. The perimeter bonding surface 19 may or may not be modified in accordance with the step of modifying 110 of the method 100 described above. In FIG. 4D, the adhesive system bond 72a, 70a bonds the microarray die 10 to the attachment surface 57 of the package 50 at the bonding surface 17, which is opposite to the array surface 16. Moreover, the attachment surfaces 47, 67, 57 of the package 40, 50, 60 may or may not be treated in accordance with the step of treating 120 of the method 100. However, in FIG. 4B, the adhesive bond 70a functions both to bond the microarray die 10, 20 to the package 40 and to seal the interface between the microarray die 10, 20 and the package frame 42 to render the apparatus 200 fluid-tight at the interface. The same is true for the package frame 62 of the package type 60. A magnified cross-sectional view of the adhesive system bond and seal 72a, 70a at the interface between the microarray die 10, 20 and the package frames 42, 62 of the packages 40, 60 is illustrated in FIG. 4C.

In some embodiments of the apparatus 200 associated with the package type 50 not illustrated in FIG. 4D, the adhesive bond 72a may extend between the microarray and the package only along the perimeter portion of the bonding surface 17. By limiting the adhesive bond 72a to the perimeter portion, noise-related signals that may occur during a subsequent scan of the microarray surface 16 are reduced.

The bond and seal 72a, 70a between the microarray die 10 and the package 40, 60 illustrated in FIGS. 4B and 4C has been demonstrated to withstand temperature extreme swings in excess of 70° C. for a microarray die 10 substrate size 18″ less than or equal to about 22×22 mm, which is believed to be due to the use of two different adhesives for the attachment and seal 72a, 70a and the use of surface treatments and modifications, according to a preferred embodiment. The package adhesive readily wets or adheres to the optionally surface treated plastic to provide sufficient bond strength to the adhesive system bond 72a, 70a after the package adhesive is cured. However, the package adhesive alone may not have sufficient flexibility to withstand physical stresses associated with different expansion rates of the different microarray and package materials during an assay. The periphery adhesive has sufficient flexibility to withstand the physical stresses, and further wets or adheres to the microarray and the package and especially to the cured package adhesive to provide sufficient bond strength to the adhesive system bond 72a, 70a. The periphery adhesive has 300 to 500% elongation before failure in a conventional bond strength test.

A single adhesive that has both a very large elongation capability and reliable bondability to both glass and plastic substrates may not be readily obtainable. Therefore, the method of attaching 130a solves this problem by using two adhesive applications to attach the microarray to the package. Using at least two adhesive applications was found to be a good technique to achieve both improved bondability and flexibility characteristics with currently available adhesives, especially for the preferred package types 40, 60, where a flexible seal is desirable during an assay that includes temperature extremes. However, if obtainable, the single adhesive that has these characteristics is within the scope of the present invention.

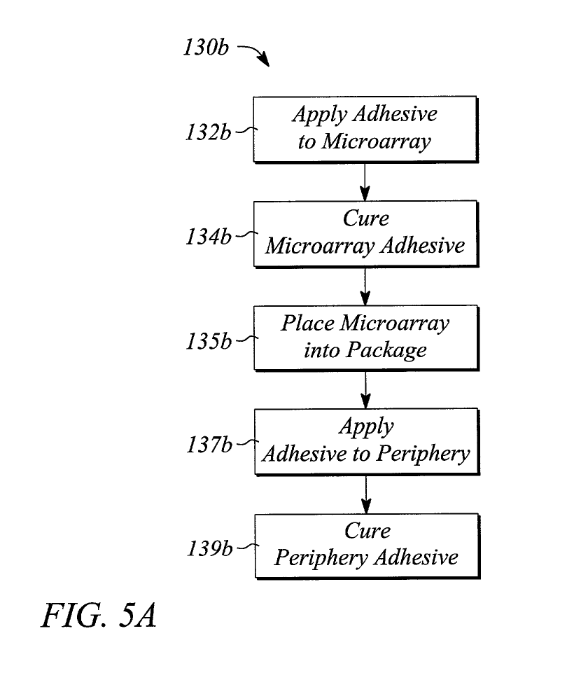

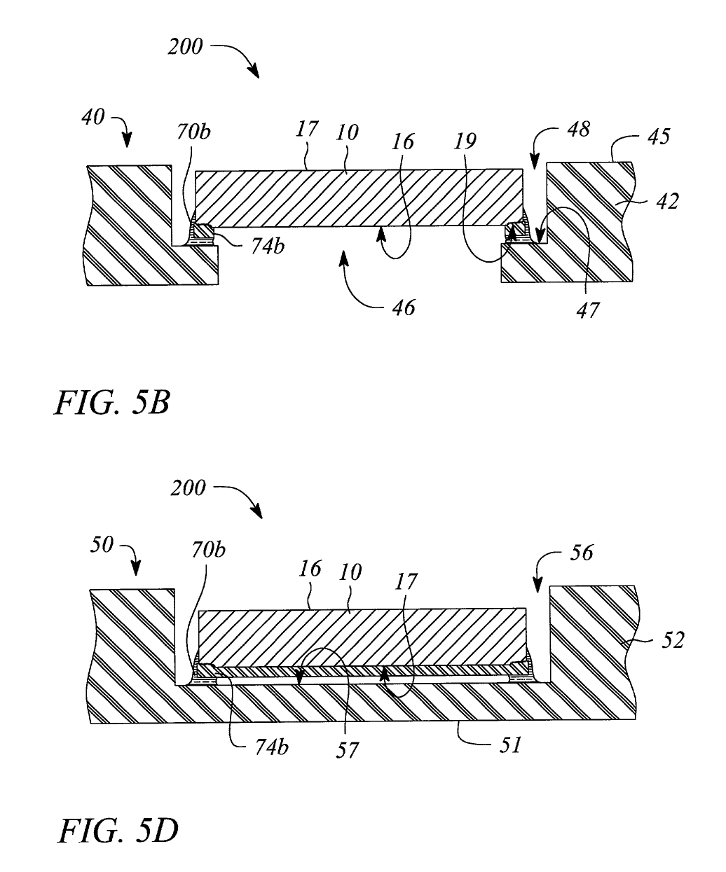

The present invention includes various alternative embodiments to the first embodiment of the method of attaching 130a using two or more adhesives. One alternative is a second embodiment of the method of attaching 130b illustrated in FIG. 5A. In the second embodiment, the method of attaching 130b the microarray comprises applying 132b a first or ‘microarray’ adhesive to the bonding surface of the microarray die, and curing 134b the microarray adhesive into a cured microarray adhesive layer 74b bonded to the microarray.

The method of attaching 130b further comprises placing 135b the microarray on the package, such that the cured microarray adhesive layer makes contact with the attachment surface of the package. The method of attaching 130b still further comprises applying 137b a second or ‘periphery’ adhesive to the periphery of the microarray die adjacent the interface between the attachment surface of the package and the cured microarray adhesive layer. The periphery adhesive wicks around the periphery of the die and into the interface between the package attachment surface and the cured microarray adhesive layer on the bonding surface. The method of attaching 130b then further comprises curing 139b the periphery adhesive to form a periphery bond 70b that bonds together the microarray die and the package at the bonding surface and the attachment surface. Either or both of the microarray bonding surface and the package attachment surface may be optionally treated or modified, as described above for the method 100.

FIGS. 5B and 5D illustrate in cross section the packaged microarray apparatus 200 comprising the microarray die 10, 20 the package 40 and 50, respectively, and an adhesive system bond 74b, 70b, according to the second embodiment. In FIG. 5B, the preferred package type 40 is illustrated and the adhesive system bond 74b, 70b provide a strong and reliable bond between the microarray die 10, 20 and the package 40. The adhesive bond 70b further seals the interface between the microarray 10, 20 and the package frame 42 to make the apparatus 200 fluid tight at the interface. The cross-sectional view in FIG. 5B also is indicative of the adhesive bond and seal 74b, 70b between the microarray 10, 20 and the package frame 62 of the package 60 on the non-recessed attachment surface 67 (not illustrated therein). A magnified cross-sectional view of the adhesive system bond 74b, 70b at the interface between the microarray and the package frames 42, 62 of the package types 40, 60 is illustrated in FIG. 5C.

In FIG. 5D, the package type 50 is used and the adhesive system bond 74b, 70b forms a reliable and strong bond between the microarray 10 and the package 50. As mentioned above for the embodiments illustrated in FIGS. 3D and 4D, in some embodiments of the apparatus 200 (not illustrated in FIG. 5D), the adhesive bond 74b may be limited to a perimeter portion of the bonding surface 17 to reduce noise-related signals during a scan of the microarray surface 16.

The microarray adhesive used in the second embodiment of the method of attaching 130b may be the same as the package adhesive from the first embodiment, or a different adhesive may be used. The microarray adhesive readily wets or adheres to the microarray bonding surface to provide sufficient bond strength to the adhesive system bond 74b, 70b after it is cured. However, the microarray adhesive alone may not have sufficient flexibility to withstand physical stresses associated with differing expansion rates of the microarray and package materials during temperature extremes. The periphery adhesive is much more flexible than the microarray adhesive, and as mentioned above, the periphery adhesive readily wets or adheres to other cured adhesives, especially the cured microarray adhesive, and adheres to both the microarray and the package with sufficient bond strength and flexibility, such that the adhesive system bond 74b, 70b will withstand the physical stresses mentioned above.

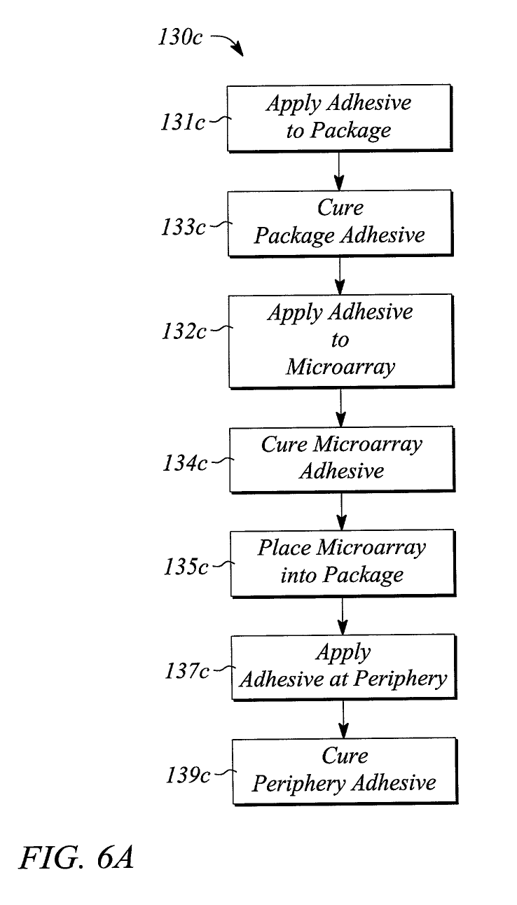

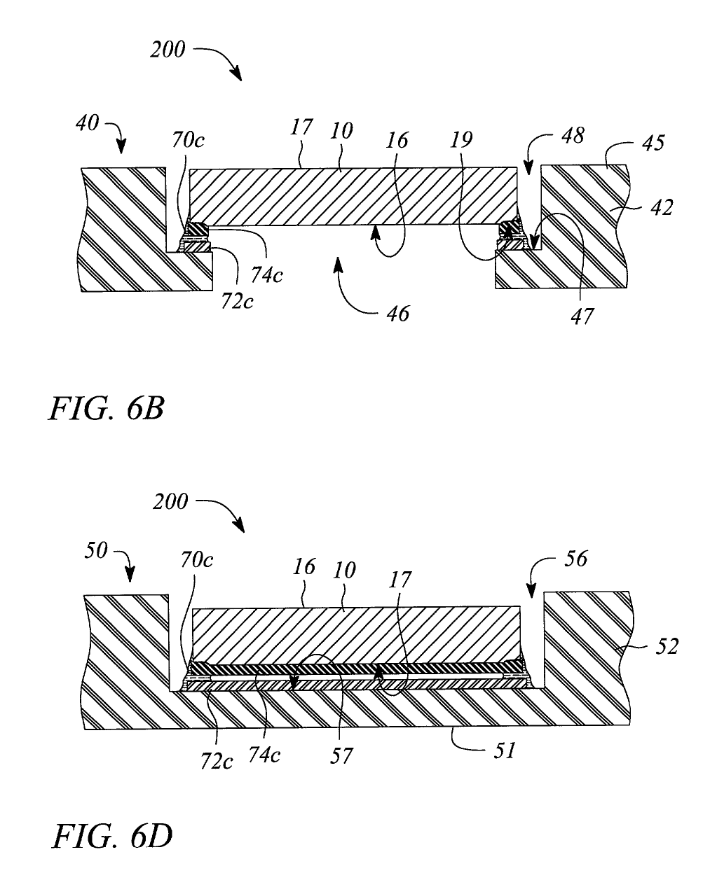

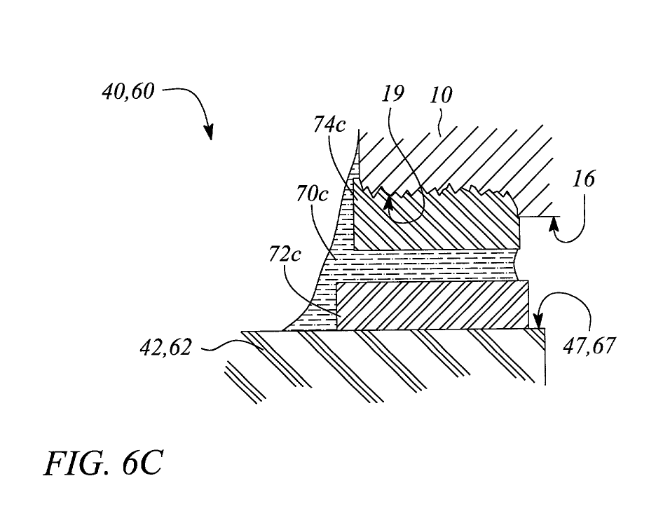

Another alternative is a third embodiment of the method of attaching 130c that is illustrated in FIG. 6A. In the third embodiment, the method of attaching 130c the microarray comprises applying 131c a first or ‘package’ adhesive to the attachment surface of the package, and curing 133c the package adhesive into a cured package adhesive layer 72c. The cured package adhesive layer preferably is uniformly thin on the attachment surface. These steps are the same as the steps 131a and 133a in the first embodiment of the method of attaching 130a described above.

The method of attaching 130c further comprises applying 132c a ‘third’ or ‘microarray’ adhesive to the bonding surface of the microarray, and curing 134c the microarray adhesive into a cured microarray adhesive layer 74c. The cured microarray adhesive layer preferably is uniformly thin on the bonding surface. These steps are similar to the steps 132b, 134b of the second embodiment of the method of attaching 130b.

The method of attaching 130c still further comprises placing 135c the microarray die into the package aligned over the cured package adhesive layer with the cured microarray adhesive layer being adjacent to the cured package adhesive layer. The method of attaching 130c further comprises applying 137c a second or ‘periphery’ adhesive to the periphery of the microarray die adjacent the cured adhesive layers. In this embodiment, the periphery adhesive wicks in between the cured adhesive layer on the bonding surface and the cured adhesive layer on the attachment surface of the package. The method of attaching 130c then further comprises curing 139c the periphery adhesive to form a periphery bond 70c. The steps of applying and curing 137c, 139c are essentially the same as the steps 137a, 137b and 139a, 139b, respectively, of the method of attaching 130a, 130bof the first and second embodiments.

Moreover, in this third embodiment, the microarray adhesive may be the same as the package adhesive or may be a different adhesive. The periphery adhesive is much more flexible than the package adhesive and the microarray adhesive and as mentioned above, readily wets or adheres to other cured adhesives and wets or adheres to the microarray and the package. None of the adhesives alone have sufficient bond strength and flexibility to withstand the physical stresses associated with different thermal expansion rates of the microarray and the package during temperature extremes. Advantageously, the adhesive system bond 72c, 74c, 70c of the method 130c does have sufficient bond strength and flexibility to withstand these stresses. As mentioned above for the other alternate embodiments 130a, 130b, in the method of attaching 130c, either or both of the microarray bonding surface and the package attachment surface may be optionally modified or treated as described above for the method 100.

FIGS. 6B and 6D illustrate cross sectional views of the packaged microarray apparatus 200 having a microarray die 10, 20, a package 40, 50 and an adhesive system bond 72c, 74c, 70c formed according to the third embodiment of the method of attaching 130c. FIG. 6B illustrates the apparatus 200 using the preferred package type 40. FIG. 6B is illustrative of the apparatus 200 using the package 60, as well, wherein the attachment surface 67 is not recessed. When package 40, 60 is used, the adhesive bond 70c further seals the interface between the microarray 10, 20 and the package frame 42, 62 to make the apparatus 200 fluid tight at the interface. A magnified cross-sectional view of the adhesive system bond 72c, 74c, 70c at the microarray/package interface of the packaged microarray apparatus 200 using either of the package types 40, 60 is illustrated in FIG. 6C. Further, for the embodiment of the packaged microarray apparatus 200 in FIGS. 6B-6D, either or both of the microarray bonding surface and the package attachment surface are optionally modified or treated surfaces.

FIG. 6D illustrates the apparatus 200 when the package type 50 is used. The apparatus 200 embodiment illustrated in FIG. 6D has a continuous adhesive layer 72c, 74c on the bonding surface 17 and attachment surface 57. However, in some embodiments using the package type 50 not illustrated herein, the microarray adhesive is applied only at the perimeter portion and the package adhesive is applied to the attachment surface only in alignment with the perimeter portion of the bonding surface 17. In these embodiments, the adhesive bonds 72c, 74c are similar in their extent to that illustrated for the periphery bond 70c between the microarray 10, 20 and the package 50 surfaces in FIG. 6D.

When an adhesive is applied to the bonding surface of the microarray die after its is populated with biological features, as in some embodiments of the step 132b, 132c of the second and third alternative embodiments of the method 130b and 130c, the method of attaching is more risky, since the method requires additional handling of the populated microarray and further manipulation of the microarray 10, 20 to apply and cure the adhesive. While these alternative embodiments provide the desired adhesive system bond according to the present invention, they are not preferred when the microarray is already populated with features. The first embodiment is preferred because it provides the least amount of handling and manipulation of the populated microarray before it is bonded to the package.

In the method of attaching 130a, 130b, 130c the microarray to the package, the package and microarray adhesives are adhesives that readily bond to a respective one or both of the optionally modified or treated glass and plastic surfaces. The periphery adhesive is an adhesive that bonds to both the glass and plastic surfaces and further readily bonds to other cured adhesives. Both time and temperature cure type epoxies and UV cure epoxies may be used for the package and microarray adhesives, and preferably UV cure epoxies are used. Examples of epoxies useful for the invention are Dymax UV cure epoxies 3011 manufactured by Dymax Corporation of Torrington, Conn.; 3M DP 460 and 3M DP-190, both of 3M Corporation, Minn.; and Loctite U-10FL of Loctite Corporation, Cleveland, Ohio. These epoxies bond readily to both of the optionally surface treated/modified glass and plastic, but they lack much flexibility.

In the step of applying 131a, 131c the package adhesive to the attachment surface of the package, the term ‘readily bonds’ or ‘readily wets or adheres’ means that the adhesive is applied such that it wets and spreads into a uniformly thin layer on the surface. Preferably, the uniform thin layer is very thin, which may be defined as a thickness of about 0.0002 to 0.015 inches (0.005 mm to 0.38 mm), preferably about 0.0005 to 0.005 inches (0.013 mm to 0.13 mm). Advantageously, the step of applying 131a, 131c of the first and third embodiments also demonstrates the integrity of the package surface or its optional treatment (step 120) prior to committing a microarray to that package in step 135a, 135c. If the package surface does not have sufficient surface energy, or if the optional surface treatment is not correct or complete, the package adhesive will not wet and spread properly as a uniformly thin layer.

The UV cure epoxy adhesives are cured 133a, 133c, 134b, 134c by exposure to UV light per the manufacturer's instructions. The time and temperature epoxy adhesives are cured with time and temperature according to the manufacturer's instructions. However, for the time/temperature cure epoxies, one skilled in the art recognizes that there are temperature limitations on the plastic used for the package and on the biological material that populates the microarray. Each plastic has its own limit and the temperature limitations of each plastic can be found in plastics handbooks known to those skilled in the art. DNA features can certainly withstand 100° C. However, if the microarray is formed of protein features before it is attached to the package, the temperature limit may be in the range of 37° C. to 40° C. to prevent protein denaturing. For UV cure epoxies, one skilled in the art further recognizes that the array surface is shielded during the UV cure, so that the biological material, if present, is not cross linked unintentionally during the adhesive cure process.

Moreover, both time and temperature cure type epoxies and acrylic urethanes and UV cure type acrylic-urethanes may be used for the periphery adhesive, and preferably UV cure acrylic-urethanes are used. For example, Dymax UV cure acrylic-urethanes manufactured by Dymax Corp. (mentioned above) have moderate bonding (i.e., bond strength) to the glass and plastic materials, and are extremely flexible. Examples of acrylic-urethane adhesives with a high amount of flexibility include: Dymax 202CTH, Dymax 3089-T, Dymax 3089 and Dymax 1-20581. In the step of applying 137a, 137b, 137c the periphery adhesive to the periphery of the microarray, after the microarray is placed 135a, 135b, 135c, the periphery adhesive is applied around the die periphery and allowed to wick into the interface.

The interface comprises the microarray bonding surface and the cured package adhesive layer 72a in the first embodiment (step 137a). The interface comprises the cured microarray adhesive layer 74b and the package attachment surface in the second embodiment (step 137b). The interface comprises the cured package and the cured microarray adhesive layers 72c, 74c on both the microarray bonding and the package attachment surfaces in the third embodiment (step 137c). The periphery adhesive is cured 139a, 139b, 139c once an evenly or uniformly thin layer has wicked around the perimeter. For the package types 40 and 60, the periphery adhesive must wick around the entire perimeter to seal the interface and render the interface fluid tight. For the package type 50, it is not necessary to achieve a fluid tight seal around the entire perimeter. The UV cure acrylic-urethane adhesives are cured 139a, 139b, 139c by exposure to UV light per the manufacturer's instructions. The time and temperature epoxy and acrylic-urethane adhesives are cured with time and temperature according to the manufacturer's instructions, taking into consideration the temperature limitations of the plastic and biological material mentioned above.

In the preferred embodiment, the UV cure or time/temperature cure epoxies provide a very good strength but rigid bond to the glass and/or plastic. The bond strength is very high as measured by conventional pull test equipment. However, the adhesive material is termed ‘rigid’ since it will only stretch to a few percent under load before it fails. The UV cure acrylic-urethanes provide relatively moderate strength, but very flexible, bonds to the glass, plastic and other cured adhesives. These adhesives will stretch 100% to 500% of their original length before shearing. The dimension of interest in this shear load application is the thickness of the adhesive. This combination of bondability and flexibility of the combination of adhesive applications of the adhesive system provides the attachment that is sufficient to withstand temperature extremes, especially during a hybridization assay. For the purposes of the present invention, either of the first, the second or the third adhesive of the combination is more flexible than the others. Preferably, the second or periphery adhesive is more flexible than the first and third adhesives.

According to the invention, the combination of adhesives used provide the bondability and also the flexibility that are sufficient enough to withstand the stresses caused by the different expansion rates of the materials during the temperature extremes. The ability to withstand the stresses due to temperature extremes is particularly important for the packaged microarray apparatus 200 that uses the preferred package types 40, 60, where the adhesive system bond also provides a fluid tight seal.

Table 1 lists the thermal expansion coefficients for glass, plastic and two UV cure adhesives that would work for the present invention. The adhesives listed in Table 1 are exemplary only and not intended to limit the scope of the present invention. Any adhesive mentioned above, or that provides the features described above, will work for the invention. One skilled in the art can readily obtain information on the characteristics of an adhesive from its manufacturer and without undue experimentation. For example, a single adhesive that provides sufficient bondability and flexibility is also desirable for the invention, especially for some embodiments of the method 100 and the apparatus 200. Alternatively, two or more adhesives that provide a thermal coefficient of expansion gradient between the different expansion coefficients of the glass and the plastic would also work for the package 40, 60, as long as the adhesives provide sufficient bondability to the glass and the plastic materials and to each other and the gradient thus formed sufficiently replaces the desired flexibility.

TABLE 1
Characteristics of Materials Useful for the Invention

| | | TCE | Flexibility = | |
|---|---|---|---|---|
| | Adhesive | (× 10⁶ per | elongation at | |
| Material | Use | ° C.) | break % | Manufacturer |
| Glass | | 3.2 | Rigid | N/A |
| Poly- | | 50-100 | 100% | N/A |
| propylene | | | | |
| ABS | | 40-100 | 3-75% | N/A |
| UV Cure | Package or | 400 | Elongation at | Dymax Corp. |
| Epoxy No. | Microarray | | break = 20% | |
| 3011 | Adhesive | | Modulus of | |
| | | | Elasticity = | |
| | | | 27,000 psi | |
| UV Cure | Periphery | 170 | Elongation at | Dymax Corp. |
| Acrylic- | Adhesive | | break = 500% | |
| urethane No. | | | Modulus of | |
| 202CTH | | | Elasticity = | |
| | | | 400 psi | |

In another aspect of the invention, a kit is provided that comprises a packaged microarray apparatus 200 in accordance with any of the embodiments, as described above, and a sample solution of biological material to use as an assay control. The kit optionally further comprises instructions for using the packaged microarray apparatus 200 in an assay. The apparatus 200 can withstand conventional temperature extremes, such as those extremes during a hybridization assay.

When the user receives the kit of the present invention, the user or an agent thereof (including but not limited to, a parent or a subsidiary of the parent or of the user, a contractor, subcontractor, vendor, customer, or the like) will typically assay a DNA, RNA or protein sample to the packaged microarray in accordance with the provided instructions. The hybridized array is then interrogated following exposure. Interrogation is usually accomplished by a suitable scanner that can read the location and intensity of fluorescence at each feature of an array following exposure to a fluorescently labeled sample (such as a polynucleotide containing sample). For example, such a scanner may be similar to the DNA Microarray Scanner available from Agilent Technologies, Palo Alto, Calif. Results from the interrogation can be processed results, such as obtained by rejecting a reading for a feature which is below a predetermined threshold and/or forming conclusions based on the pattern read from the array (such as whether or not a particular target sequence may have been present). The hybridization assay may be performed in a first location, the interrogation may be performed in the first or a second location different from the first by a user or an agent thereof, and the results of the interrogation (processed or not) can be forwarded (such as by communication) to a third location remote from the first or second location, if desired, and received there for further use by the user or an agent thereof.

By “remote” location, it is meant that the first, second or third location is at least in a different building from the others, and may be at least one mile, ten miles, or at least one hundred miles apart. “Communicating” information references transmitting the data representing that information as electrical signals over a suitable communication channel (for example, a private or public network). “Forwarding” an item (including information) refers to any means of getting that item from one location to the next, whether by physically transporting that item or otherwise (where that is possible) and includes, at least in the case of data, physically transporting a medium carrying the data or communicating the data.

For the purposes of the present invention, the biological material bound to the microarray surface may be a nucleic acid, protein, polypeptide, polysaccharide, ligand, receptor, antigen, antibody, or the like, either as a probe or a target under test. Further, the other complementary material may be either the probe or the target. However, when the probe is bound to the microarray, the other material is the target and where the target is bound to the microarray, the other material is the probe. Further, the target material or the probes may be directly or indirectly labeled using conventional methods, such that after hybridization, the targets or probes that are hybridized either emit a signal when optically interrogated or have detectable radioactivity. The resulting hybridized microarray of the invention is washed to flush unhybridized and non-specifically hybridized material off the surface and then spun or blown dry using conventional methods. In some embodiments, the dried microarray is optically scanned to measure the degree of hybridization using conventional scanning equipment and techniques. Where the target or probe is indirectly labeled, a post-hybridization stain, such as streptavidin covalently linked to a fluorophore or colloidal gold, is typically applied to the microarray after hybridization, but before final washing. The signal intensities and locations of the signals on the microarray provide much information about the material being evaluated.

### EXAMPLES

### Adhesive Temperature Cycling Example

Several packages 40 consisting of a frame 42 and sidewall portion 44 were injection molded from polypropylene. The frame 42 and sidewall portion 44 were ultrasonically welded together. The frame 42 comprised an opening 46 that was 0.0800″ (20.3 mm) square. A thin ledge or recessed surface 47 surrounded the opening 46, which was sized to accept a 22×22 mm glass die. This ledge 47 was surface treated for 20 seconds by an electrical discharge system (Tantec Low Frequency Spot Generator HP-S). Treatment was performed on each package with the electrode placed at a distance of 0.5″-2.5″ above the attachment surface for a minimum of 20 seconds. The angle of the corona arc discharge was varied in relation to the surface that was treated. A package adhesive, DP-460 (3M, Minneapolis, Minn.) 2-part epoxy, was mixed per the manufacturer's instructions and applied in a very thin layer to each treated ledge surface 47. The epoxy was cured at 60° C. for several hours.

One-millimeter thick glass substrates 18 were processed to obtain a hydrophobic surface appropriate for in-situ synthesis. However, no synthesis was performed to save on cost for this Example. Each substrate 18 was diced into to 22×22 mm die using a Disco dicing saw. As the substrate was diced, peripheries of the individual die were simultaneously ground down about 25-250 micron for a perimeter 19 width of 0.75 mm on the hydrophobic surface. The adhesive, 3M DP-460, 2-part epoxy, was mixed per the manufacturer's instructions and applied in a very thin layer to the ground periphery 19 of each die. The epoxy was cured at 60° C. for several hours.

One die was placed into the recess 48 of each welded package 40, such that the cured epoxy layers faced each other. A Dymax 3089T UV curable acrylic-urethane was applied to the periphery of each die and allowed to wick between the two cured epoxy layers. The periphery adhesive was cured using a Dymax 3010EC Spot Curing Lamp.

To test the integrity of the assembly, each package was placed into a 70° C. incubator for 17 hours. After 17 hours, the packages were moved to a −20° C. freezer for an hour. After the hour, the packages were returned to the 70° C. incubator for an hour. The packages were cycled back through the −20° C. freezer and the 70° C. incubator for an hour each. The temperature extremes chosen were ranges that simulate temperature extremes of an assay and further that attempt to simulate temperature extremes to which a packaged microarray might be exposed while being shipped to a user. After this treatment cycle, the comers of the adhesive showed minor stress marks, but the seal at the interface between the die and the package was intact and fluid tight. The intact seal was determined to be intact and fluid tight visually and by applying a colored test fluid to the seal area. Intact seals had >90% fluid retention and were considered to be fluid tight for the purposes of this Example. Fluid loss was due to water vapor retention and/or through the interface between the frame 42 and sidewall portion 44 of the package type 40 when the package was exposed to 70° C. for 17 hours.

### Hybridization Example

Several packages 40 consisting of a frame 42, sidewall portion 44 and lid 43 were injection molded from polypropylene. The frame 42 and sidewall portion 44 were ultrasonically welded together. The frame 42 comprised an opening 46 that was 0.800″ (20.3 mm) square. A thin ledge 47 surrounded the opening 46, which was sized to accept a 22×22 mm glass die. This ledge 47 was surface treated for 20 seconds by an electrical discharge system (Tantec Low Frequency Spot Generator HP-S), similar to the Example described above. The lid 43 was a removable package lid with septa installed. The lid 43 was snapped into each welded assembly (see description in U.S. Pat. No. 6,261,523). A package adhesive, Dymax 3011 UV Cure Epoxy was applied to the surface treated ledge 47 along the periphery of each frame opening 46 using an Asymtek adhesive dispensing system with x, y stage control. The Dymax 3011 epoxy was cured using a Dymax EC2000 flood lamp for 20-40 seconds.

In-situ synthesized 25-mer oligonucleotide arrays containing 8455 features were fabricated by appropriate procedures on 1-mm thick glass substrates that had chromed fiducial and die reference numbers visible and a hydrophobic surface designed for DNA in-situ synthesis. The arrays contained 4×-replicated, optimized probes against human reference sequence genes (RefSeq). Three sequences were used per gene, for a total of 12 probes per gene. Each array included a variety of positive and negative control probes. The probes were arranged into 4 identical quadrants on the glass substrates. The populated substrates were diced into 22×22 mm die 10 using a Disco dicer. As they were diced, the perimeter 19 of the die 10 were simultaneously ground down 25-250 micron for a perimeter width of 0.75 mm on the same surface as the oligomer arrays.

Each populated and bonding surface-modified die 10 was placed onto the ledge 47 of opening 46 in a welded package assembly 40. The array 12 was oriented such that the array 12 faced into the package 40 and the ground perimeter surface 19 faced the cured epoxy on the ledge 47. The array 12 was also oriented so that the die number was in the lower right corner to ensure that the array 12 was properly oriented for subsequent scanning and feature extraction.

Each package 40 with a corresponding die 10 in place was put into the Asymtek 403 Benchtop adhesive application equipment. An adhesive, Dymax 202CTH acrylic-urethane adhesive, was dispensed around the periphery of the die 10 and allowed to wick between the die 10 and the ledge 47 of the package 40. The periphery adhesive was cured using the Dymax EC2000 flood lamp for 20-40 seconds.

A bar code describing the array design was placed on each packaged array apparatus 200 thus assembled. A fluorescently labeled mRNA sample from K562 cells was injected into each packaged array 200. The sample quantity was 250 μL. The packaged arrays were hybridized by incubation overnight at 60° C. During incubation, the contents of the packaged arrays were bubble mixed by package rotation. After the hybridization, the array was still immersed in sample indicating that the seal between the frame 42 and the array substrate 10 was intact. Next, the package lid 43 was removed and the arrays were washed in 6× SSC (0.9 m Sodium Chloride and 0.09M Sodium Citrate, available from Amresco, Solon, Ohio) at room temperature for 10 minutes. Next the packaged arrays were washed in 0.1× SSC (0.015M Sodium Chloride and 0.0015M Sodium Citrate, available from Amresco, Solon, Ohio) on ice for 5 minutes. They were dried by centrifugation. The substrates were inspected after the wash and dry processes and they remained firmly attached to the package. The packaged arrays were scanned in a fluorescent scanner manufactured by Agilent Technologies. The data extracted from the arrays were equivalent to similar arrays that were manually hybridized as a control.

Thus there has been described a new method 100 of bonding microarrays into a package, a method 130a-130c of attaching a populated microarray to a package, a packaged microarray apparatus 200 and a kit for using a packaged microarray apparatus 200 in an assay, according to various embodiments. It should be understood that the above-described embodiments and examples are merely illustrative of some of the many specific embodiments that represent the principles of the present invention. Clearly, numerous other arrangements can be readily devised by those skilled in the art without departing from the scope of the present invention.

What is claimed is:

1. A method of bonding a microarray to a microarray package comprising:
modifying a bonding surface of the microarray so as to improve bonding of an adhesive to the bonding surface; and
attaching the microarray to the microarray package using the adhesive between the modified bonding surface and an attachment surface of the package.

2. The method of claim 1, wherein after the bonding surface is modified, further comprising attaching biological features to an array surface of the microarray.

3. The method of claim 1, wherein before the bonding surface is modified, further comprising attaching biological features to an array surface of the microarray.

4. The method of claim 1, wherein the bonding surface is a surface perimeter of the microarray.

5. The method of claim 4, wherein the bonding surface of the microarray is modified by removing a surface layer from the bonding surface perimeter.

6. The method of claim 5, wherein the step of removing a surface layer comprises:
dicing an array substrate into a plurality of individual microarrays; and
grinding the bonding surface perimeter.

7. The method of claim 6, wherein the step of grinding is performed simultaneously with the step of dicing.

8. The method of claim 6, wherein the surface layer is removed using at least two blades, a first blade being wider than a second blade, wherein the step of dicing comprises using the second blade to dice the array substrate, and wherein the step of grinding comprises using the first blade to remove the surface layer from the bonding surface perimeter.

9. The method of claim 1, wherein the bonding surface has a surface energy, and the bonding surface of the microarray is modified by treating a perimeter of the bonding surface to change the surface energy relative to the surface energy before the treatment.

10. The method of claim 4, wherein the bonding surface perimeter of the microarray is modified by masking a surface of the microarray except the perimeter; and exposing the unmasked perimeter to one of corona or plasma.

11. The method of claim 4, wherein the bonding surface perimeter of the microarray is modified by masking the perimeter; and exposing an unmasked surface to treatment suitable for bonding biological features to the treated surface.

12. The method of claim 1, further comprising treating the attachment surface of the microarray package so as to improve bonding of the adhesive to the attachment surface.

13. The method of claim 12, wherein the attachment surface of the microarray package is treated by one or more of plasma etching a surface layer from the attachment surface, and exposing a surface layer of the attachment surface to corona.

14. The method of claim 12, wherein the attachment surface has a surface energy, and the attachment surface of the microarray package is treated to change the surface energy of a surface layer of the attachment surface by using one or more of electrical discharge and fluorine surface treatment, wherein the surface energy of the surface layer of the attachment surface is changed relative to before the treatment.

15. The method of claim 1, wherein the adhesive that is used to attach the microarray to the package forms a bond between the microarray and the package when cured, the bond having bond strength and flexibility that are sufficient enough that the bond provides a fluid tight seal with physical stresses caused by different thermal expansion rates of the microarray and the package.

16. The method of claim 15, wherein the adhesive comprises a plurality of separate adhesive applications, and wherein the bond comprises multiple adhesive bonds, the multiple adhesive bonds being stronger and more flexible than any one of the bonds formed by a respective one adhesive application of the plurality alone to attach the microarray to the package.

17. A method of attaching a microarray to a microarray package comprising:
using a combination of adhesives between a bonding surface of the microarray and an attachment surface of the package to form an adhesive bond, the combination of adhesives providing more bond strength and flexibility to the adhesive bond relative to a bond formed by any one of the adhesives used individually to bond the microarray and the package.

18. The method of claim 17, wherein the adhesive combination comprises a cured first adhesive bonded to either or separately to both of the microarray bonding surface and the package attachment surface, and a cured second adhesive that joins the microarray and the package.

19. The method of claim 18, wherein one of the cured first adhesive and the cured second adhesive is more flexible than the other.

20. The method of claim 17, wherein the adhesive bond has the bond strength and the flexibility that are sufficient enough to provide a fluid tight seal with physical stresses caused by different thermal expansion rates of the microarray and the package.

21. The method of claim 17, further comprising either or both of modifying the microarray bonding surface and treating the package attachment surface, so as to improve bonding of the adhesives to the respective surface.

22. The method of claim 17, wherein the adhesive combination comprises a periphery adhesive, and one or both of a microarray adhesive cured to the bonding surface of the microarray and a package adhesive cured to the attachment surface of the package, the periphery adhesive being more flexible than either of the microarray adhesive and the package adhesive, and wherein the adhesive bond comprises the periphery adhesive cured to the package, to the microarray, and either or both of the cured microarray adhesive and the cured package adhesive.

23. The method of claim 22, wherein the microarray adhesive is the same as the package adhesive.

24. The method of claim 17, wherein the step of using a combination of adhesives comprises:
applying a package adhesive to the attachment surface of the package, and curing the package adhesive to form a cured package adhesive layer;
placing the microarray into the package such that the bonding surface of the microarray is aligned with the cured package adhesive layer; and
applying a periphery adhesive to a periphery of the microarray adjacent to an interface between the bonding surface of the microarray and the cured package adhesive layer of the package attachment surface, and curing the periphery adhesive to form the adhesive bond between the microarray and the package, wherein the periphery adhesive is allowed to wick into the interface before the periphery adhesive is cured.

25. The method of claim 17, wherein the step of using a combination of adhesives comprises:
applying a microarray adhesive to the bonding surface of the microarray, and curing the microarray adhesive into a cured microarray adhesive layer;
placing the microarray into the package, such that the cured microarray adhesive layer on the bonding surface is adjacent to the attachment surface of the package; and
applying a periphery adhesive to a periphery of the microarray adjacent to an interface between the cured microarray adhesive layer and the attachment surface, and curing the periphery adhesive to form the adhesive bond between the microarray and the package, wherein the periphery adhesive is allowed to wick into the interface before the periphery adhesive is cured.

26. The method of claim 24, before the step of placing, further comprising:
applying a microarray adhesive to the bonding surface of the microarray, and curing the microarray adhesive into a cured microarray adhesive layer,
wherein in the step of applying the periphery adhesive, the interface is between the cured microarray adhesive layer on the bonding surface and the cured package adhesive layer on the attachment surface, and the periphery adhesive wicks in between the cured package adhesive layer and the cured microarray adhesive layer at the interface before the periphery adhesive is cured.

27. A packaged microarray apparatus comprising:
a microarray having biological features attached to an array surface of a microarray substrate, the microarray comprising a modified bonding surface to improve bonding with an adhesive;
a package having an attachment surface; and
an adhesive bond between the microarray and the package at an interface between the modified bonding surface and the attachment surface.

28. The packaged microarray apparatus of claim 27, wherein the modified bonding surface of the microarray is a perimeter surface of the array surface surrounding the biological features.

29. The packaged microarray apparatus of claim 28, wherein the package comprises a frame for supporting the microarray, the frame having an interior opening and comprising the attachment surface surrounding the opening, and wherein the biological features on the array surface are aligned in the opening.

30. The packaged microarray apparatus of claim 29, wherein the adhesive bond comprises a fluid tight seal between the microarray and the frame at the attachment surface.

31. The packaged microarray apparatus of claim 27, wherein the attachment surface of the package is treated to improve bonding with the adhesive.

32. The packaged microarray apparatus of claim 27, wherein the adhesive bond comprises an epoxy cured to one or separately to both of the modified microarray bonding surface and the attachment surface, and an acrylic-urethane applied around a periphery of the microarray at an interface between the microarray and the package, such that the acrylic-urethane is wicked into the interface before it is cured.

33. A packaged microarray apparatus comprising:
a microarray having biological features attached to an array surface of a microarray substrate and having a bonding surface;
a package having an attachment surface; and
an adhesive bond between the microarray and the package, the adhesive bond comprising a combination of adhesives that provides more bond strength and flexibility than any one of the adhesives of the combination used individually to bond the microarray to the package.

34. The packaged microarray apparatus of claim 33, wherein the adhesive bond has the bond strength and the flexibility sufficient enough to provide a fluid tight seal with physical stresses from different thermal expansion rates of the microarray substrate and the package.

35. The packaged microarray apparatus of claim 33, wherein the adhesive bond comprises a cured first adhesive layer between the bonding surface and the attachment surface, and a cured second adhesive around a periphery of the microarray substrate that wicks between the bonding surface and the attachment surface adjacent to the cured first adhesive layer before being cured, and wherein the cured first adhesive layer is bonded to one or separately to both of the microarray bonding surface and the package attachment surface.

36. The packaged microarray apparatus of claim 35, wherein the adhesive bond further comprises a cured third adhesive layer between the bonding surface and the attachment surface adjacent to the cured first adhesive layer, and wherein the second adhesive wicks between the cured first adhesive layer and the cured third adhesive layer before being cured, and wherein the cured third adhesive layer is separately bonded to the other of the microarray bonding surface and the package attachment surface when the first cured adhesive layer is bonded to the one.

37. The packaged microarray apparatus of claim 33, wherein either or both of the attachment surface of the package and the bonding surface of the microarray are treated or modified to improve bonding with the adhesives of the combination.

38. The packaged microarray apparatus of claim 36, wherein the cured first adhesive is the same as the cured third adhesive.

39. The packaged microarray apparatus of claim 35, wherein the cured first adhesive layer comprises an epoxy and the cured second adhesive comprises an acrylic-urethane.

40. A method comprising:
masking a portion of a surface of a microarray; and
exposing an unmasked portion of the surface to treatment suitable for bonding biological features to the treated unmasked portion, wherein the masked portion is masked by attaching the portion to an attachment surface of a microarray package.
